(19) United States
(12) Patent Application Publication
(10) Pub. No.: US 2025/0114018 A1
(43) Pub. Date: Apr. 10, 2025

(54) DETECTION DEVICE, DETECTION SYSTEM, GAIT MEASUREMENT SYSTEM, DETECTION METHOD, AND RECORDING MEDIUM

(71) Applicant: NEC Corporation, Minato-ku, Tokyo (JP)

(72) Inventors: Yoshitaka NOZAKI, Tokyo (JP); Chenhui HUANG, Tokyo (JP); Kenichiro FUKUSHI, Tokyo (JP); Zhenwei WANG, Tokyo (JP); Fumiyuki NIHEY, Tokyo (JP); Hiroshi KAJITANI, Tokyo (JP); Kentaro NAKAHARA, Tokyo (JP)

(73) Assignee: NEC Corporation, Minato-ku, Tokyo (JP)

(21) Appl. No.: 18/729,202

(22) PCT Filed: Feb. 17, 2022
(86) PCT No.: PCT/JP2022/006330

§ 371 Date: Jul. 16, 2024

(51) Int. Cl. A61B 5/11

(52) U.S. Cl. CPC A61B 5/112

(57) ABSTRACT

A detection device that includes an acquisition unit that acquires data including a dorsiflexion peak time, a plantarflexion peak time, and travel direction acceleration acquired from sensor data regarding a movement of a foot, a candidate detection unit that detects, as a candidate time for heel strike, a time of a feature signal point extracted from time-series data of the travel direction acceleration in an investigation time period starting from an acceleration peak time detected from the travel direction acceleration based on the dorsiflexion peak time, and an output unit that outputs the detected candidate time as a heel strike time.

### TECHNICAL FIELD

The present disclosure relates to a detection device and others that detect a gait phenomenon in accordance with a gait of a user.

### BACKGROUND ART

With increasing interest in healthcare, services for providing information in accordance with features included in a gait pattern (also referred to as a gait) have attracted attention. For example, a technique for analyzing a gait based on sensor data measured by a sensor mounted on footwear such as a shoe has been developed. Features in a gait phenomenon (also referred to as a gait event) associated with a physical condition appear in time-series data of sensor data. Accurate detection of a timing of a gait event enables to highly accurately estimate a physical condition.

PTL 1 discloses a device for evaluating a foot portion and a gait. The device described in PTL 1 acquires plantar-pressure data while a user is walking or is standing up and standstill from a pressure sensor installed in a shoe used by the user. The device described in PTL 1 analyzes the acquired plantar-pressure data to acquire various types of parameters.

PTL 2 discloses a gait evaluation system. The system described in PTL 2 calculates a gait evaluation value for a subject using acceleration data in three axial directions, which has been measured by an acceleration sensor attached to an ankle of the subject.

PTL 3 discloses a movement analysis device. The device described in PTL 3 identifies a timing of a gate movement of a person using triaxial acceleration data acquired by a smartphone attached to a body trunk of the person and a movement signal acquired by a pressure sensor attached to a sole of a foot.

NPL 1 discloses a method of calculating a gait parameter using sensor data of an inertial sensor including an acceleration sensor and an angular velocity sensor. In the method described in NPL 1, a timing of a gait event of a subject and a parameter about a gait are calculated using triaxial acceleration data and triaxial angular velocity data measured by the inertial sensor attached to a side surface of a shoe.

### CITATION LIST

### Patent Literature

PTL 1: WO 2018/164157 A1
PTL 2: JP 2019-150329 A
PTL 3: JP 2015-83085 A

### Non Patent Literature

NPL 1: A. Rampp, et. al., “Inertial sensor-based stride parameter calculation from gait sequences in geriatric patients”, IEEE Transactions on Biomedical Engineering, Vol. 62, No. 4, April 2015, pp. 1089-97.

### SUMMARY OF INVENTION

### Technical Problem

In the method described in PTL 1, a gait state of the user is analyzed using data measured by the pressure sensor installed inside a shoe. In the method described in PTL 1, however, when the pressure sensor is not provided in a shoe, it has been impossible to analyze a gait state of the user.

In the method described in PTL 2, a gait state of a subject is analyzed using data measured by the acceleration sensor attached to an ankle. In the method described in PTL 2, however, when the acceleration sensor is not attached to an ankle, it has been impossible to analyze a gait state of the subject.

In the method described in PTL 3, a gait state of a person is analyzed using data measured by the acceleration sensor attached to a body trunk and the pressure sensor installed on a sole of a foot. In the method described in PTL 3, however, when a smartphone is not attached to a body trunk or when the pressure sensor is not installed on a sole of a foot, it has been impossible to analyze a gait state of the person.

In the method described in NPL 1, when an operation frequency of the acceleration sensor is 100 hertz (Hz) or more, it is possible to detect a heel strike event of a walking person. When the operation frequency of the acceleration sensor is below 100 Hz, however, a steep minimum peak is less likely to occur, and it has thus been impossible to apply the method described in NPL 1.

An object of the present disclosure is to provide a detection device and others that make it possible to detect heel strike in a gait of a user using data measured by a sensor installed on a foot portion of the user.

### Solution to Problem

A detection device according to one aspect of the present disclosure includes an acquisition unit that acquires data including a dorsiflexion peak time, a plantarflexion peak time, and travel direction acceleration acquired from sensor data regarding a movement of a foot, a candidate detection unit that detects, as a candidate time for heel strike, a time of a feature signal point extracted from time-series data of the travel direction acceleration in an investigation time period starting from an acceleration peak time detected from the travel direction acceleration based on the dorsiflexion peak time, and an output unit that outputs the detected candidate time as a heel strike time.

In a detection method according to the one aspect of the present disclosure, data including a dorsiflexion peak time, a plantarflexion peak time, and travel direction acceleration acquired from sensor data regarding a movement of a foot is acquired, a time of a feature signal point extracted from time-series data of the travel direction acceleration in an investigation time period starting from an acceleration peak time detected from the travel direction acceleration based on the dorsiflexion peak time is detected as a candidate time for heel strike, and the detected candidate time is outputted as a heel strike time.

A program according to the one aspect of the present disclosure causes a computer to execute processing for acquiring data including a dorsiflexion peak time, a plantarflexion peak time, and travel direction acceleration acquired from sensor data regarding a movement of a foot, processing for detecting, as a candidate time for heel strike, a time of a feature signal point extracted from time-series data of the travel direction acceleration in an investigation time period starting from an acceleration peak time detected from the travel direction acceleration based on the dorsiflexion peak time, and processing for outputting the detected candidate time as a heel strike time.

### Advantageous Effects of Invention

According to the present disclosure, it is possible to provide a detection device and others that make it possible to detect heel strike in a gait of a user using data measured by a sensor installed on a foot portion of the user.

### BRIEF DESCRIPTION OF DRAWINGS

FIG. 1 is a block diagram illustrating an example of a configuration of a detection system according to a first example embodiment.

FIG. 2 is a conceptual diagram for describing an arrangement example of a measurement device included in the detection system according to the first example embodiment.

FIG. 3 is a conceptual diagram for describing a coordinate system set in the measurement device included in the detection system according to the first example embodiment.

FIG. 4 is a conceptual diagram for describing a foot-portion inclination angle measured by the measurement device included in the detection system according to the first example embodiment.

FIG. 5 is a conceptual diagram for describing human body surfaces used in the description about the detection system according to the first example embodiment.

FIG. 6 is a conceptual diagram for describing a gait cycle used in the description about a gait measurement device according to the first example embodiment.

FIG. 7 is a block diagram illustrating an example of a configuration of the measurement device included in the detection system according to the first example embodiment.

FIG. 8 is a graph for describing a dorsiflexion peak and a plantarflexion peak detected by the measurement device included in the detection system according to the first example embodiment.

FIG. 9 is a block diagram illustrating an example of a configuration of a detection device included in the detection system according to the first example embodiment.

FIG. 10 is a graph for describing acceleration peaks detected by the detection device included in the detection system according to the first example embodiment.

FIG. 11 is a graph for describing a time in a mid-stance period, which is calculated by the detection device included in the detection system according to the first example embodiment.

FIG. 12 is a graph for describing an example of detecting a first candidate time by the detection device included in the detection system according to the first example embodiment.

FIG. 13 is a graph for describing an example of detecting a first candidate time by the detection device included in the detection system according to the first example embodiment.

FIG. 14 is a flowchart for describing operation of the detection device included in the detection system according to the first example embodiment.

FIG. 15 is a block diagram illustrating an example of a configuration of a detection device according to a second example embodiment.

FIG. 16 is a graph for describing an example of detecting a second candidate time by the detection device according to the second example embodiment.

FIG. 17 is a flowchart for describing operation of the detection device according to the second example embodiment.

FIG. 18 is a block diagram illustrating an example of a configuration of a detection device according to a third example embodiment.

FIG. 19 is a graph for describing an example of detecting a third candidate time by the detection device according to the third example embodiment.

FIG. 20 is a flowchart for describing operation of the detection device according to the third example embodiment.

FIG. 21 is a block diagram illustrating an example of a configuration of a detection device according to a fourth example embodiment.

FIG. 22 is a graph for describing an example of determining a heel strike time by the detection device according to the fourth example embodiment.

FIG. 23 is a flowchart for describing operation of the detection device according to the fourth example embodiment.

FIG. 24 is a block diagram illustrating an example of a configuration of a gait measurement system according to a fifth example embodiment.

FIG. 25 is a conceptual diagram for describing an application example of the gait measurement system according to the fifth example embodiment.

FIG. 26 is a block diagram illustrating an example of a configuration of a detection device according to a sixth example embodiment.

FIG. 27 is a block diagram illustrating an example of a hardware configuration for achieving processing according to each of the example embodiments.

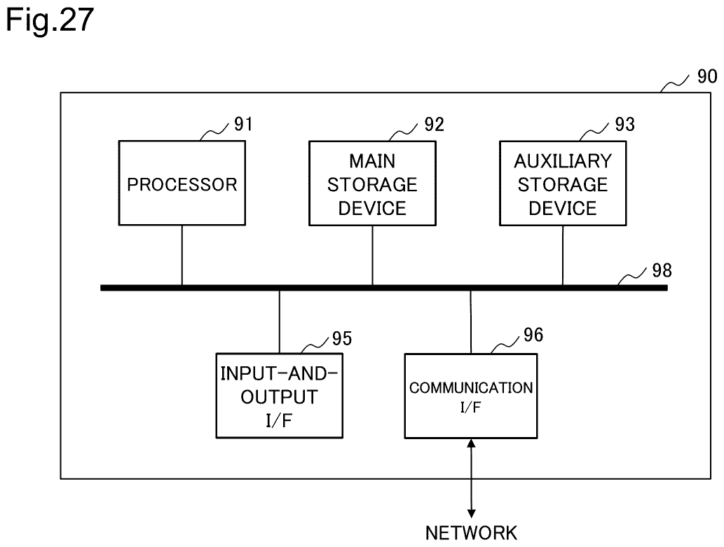

### EXAMPLE EMBODIMENT

Example embodiments of the present invention will now be described herein with reference to the accompanying drawings. Although, in the example embodiments described below, there are technically preferable limitations for carrying out the present invention, however, the scope of the invention is not limited to these described below. In all the accompanying drawings used for the example embodiments described below, identical reference numerals are assigned to similar or identical components unless there is a particular reason. In the example embodiments described below, repeated description about similar or identical configurations and operation may be omitted.

### First Example Embodiment

A detection system according to a first example embodiment will now first be described herein with reference to the accompanying drawings. The detection system according to the present example embodiment measures sensor data regarding a movement of a foot, which is measured in accordance with a gait of a user. The detection system according to the present example embodiment detects a timing of heel strike that is one phenomenon in accordance with a gait (also referred to as a gait event) from the measured sensor data. The timing of heel strike (also referred to as a heel strike time) detected by the detection system according to the present example embodiment is used, for example, for detecting another gait event and for calculating gait parameters. For example, gait parameters are used to estimate a physical condition of the user.

### (Configuration)

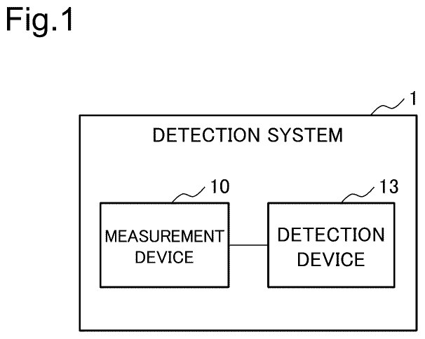

FIG. 1 is a block diagram illustrating an example of a configuration of a detection system 1 according to the present example embodiment. The detection system 1 includes a measurement device 10 and a detection device 13. The measurement device 10 and the detection device 13 may be coupled in a wired or wireless manner. The measurement device 10 and the detection device 13 may be formed in a single device.

The present example embodiment will be described with reference to an example in which the measurement device 10 and the detection device 13 are formed as separate pieces of hardware. For example, the measurement device 10 is installed on footwear of the user serving as a target for which its physical condition is to be estimated. For example, a function of the detection device 13 is installed in a mobile terminal carried by the user. The measurement device 10 and the detection device 13 may be formed in an identical piece of hardware. For example, the measurement device 10 and the detection device 13 are formed in an identical piece of hardware and installed on the footwear of the user. Configurations of the measurement device 10 and the detection device 13 will now be individually described here in.

### [Measurement Device]

The measurement device 10 is installed on a foot portion of the user. For example, the measurement device 10 is installed on the footwear of the user. The measurement device 10 measures sensor data regarding a movement of the foot. The measurement device 10 includes a sensor including an acceleration sensor and an angular velocity sensor, for example. The measurement device 10 generates sensor data using a measurement value measured by the sensor in accordance with a movement of the foot.

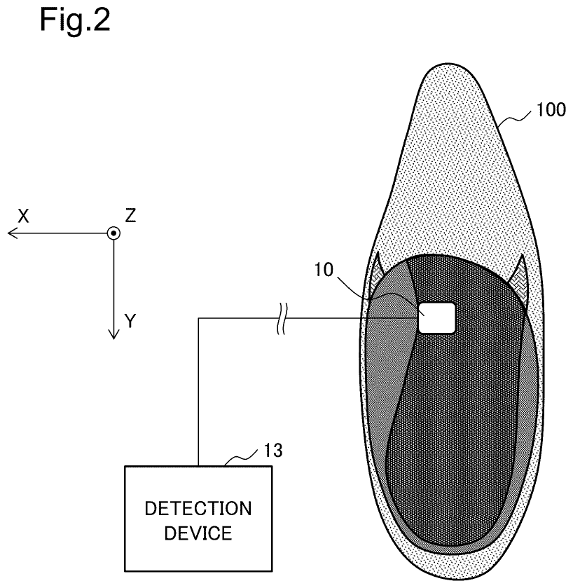

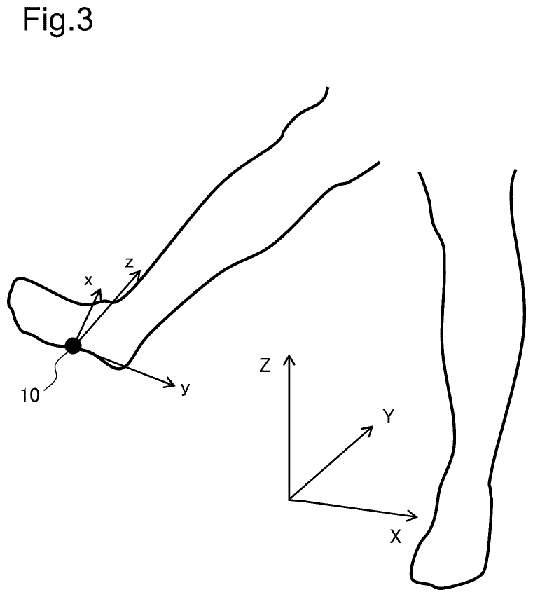

FIG. 2 is a conceptual diagram illustrating an example in which the measurement device 10 is disposed in a shoe 100 for a right foot. In the example illustrated in FIG. 2, the measurement device 10 is installed at a position with which a back side of an arch of the foot comes into contact. For example, the measurement device 10 is disposed in an insole inserted in the shoe 100. For example, the measurement device 10 may be disposed on a side surface or a bottom surface of the shoe 100. For example, the measurement device 10 may be embedded in a main body of the shoe 100. The measurement device 10 may be detachable from the shoe 100 or may not be detachable from the shoe 100. The measurement device 10 may be installed at a position other than the back side of the arch of the foot as long as it is possible to measure sensor data regarding a movement of the foot. The measurement device 10 may be installed in a sock worn by the user or a decorative article such as an anklet worn by the user. The measurement device 10 may be directly applied to the foot or may be embedded in the foot. FIG. 3 illustrates an example in which the measurement device 10 is installed on the shoe 100 for the right foot. The measurement device 10 may be installed on a shoe 100 for a left foot. The measurement device 10 may be installed on each of the shoes 100 for both the feet.

In the example illustrated in FIG. 2, a local coordinate system including an x axis in left and right directions (horizontal directions), a y axis in front and rear directions (travel directions), and a z axis in up and down directions (vertical directions) is set based on the measurement device 10. On the x axis, its leftward orientation is set as positive. On the y axis, its rearward orientation is set as positive. On the z axis, its upward orientation is set as positive. The directions of the axes set in the measurement device 10 may be identical for the left and right feet, or may be different for the left and right feet. For example, when the measurement devices 10 produced in identical specifications are disposed in the left and right shoes 100, respectively, the vertical directions (orientations in the Z-axis direction) of sensors 11 disposed in the left and right shoes 100, respectively, are identical to each other. In such a case, the three axes of the local coordinate system set for sensor data derived from the left foot and the three axes of the local coordinate system set for sensor data derived from the right foot are identical to each other on the left and right.

FIG. 3 is a conceptual diagram for describing the local coordinate system (the x axis, the y axis, and the z axis) set in the measurement device 10 installed on the back side of the arch of the foot and a world coordinate system (an X axis, a Y axis, and a Z axis) set with respect to the ground. In a state where the user is standing up and faces a front in one of the travel directions, in the world coordinate system (the X axis, the Y axis, and the Z axis), lateral directions of the user are set as X-axis directions (a leftward orientation is referred to as positive), travel directions of the user are set as Y-axis directions (a rearward orientation is referred to as positive), and directions about gravity are set as Z-axis directions (a vertically upward orientation is referred to as positive). The example illustrated in FIG. 3 conceptually illustrates a relationship between the local coordinate system (the x axis, the y axis, and the z axis) and the world coordinate system (the X axis, the Y axis, and the Z axis). The coordinate system illustrated in FIG. 3 does not accurately indicate the relationship between the world coordinate system and the local coordinate system, which changes depending on a gait of the user.

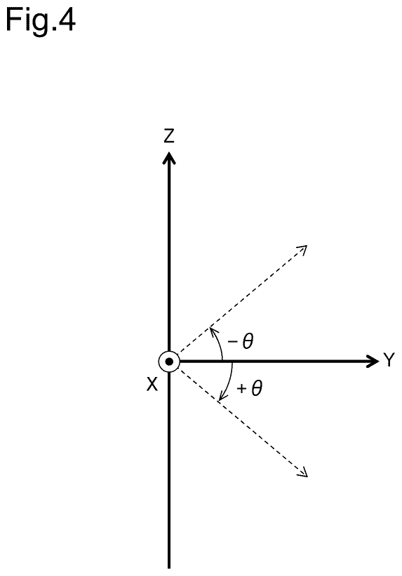

FIG. 4 is a conceptual diagram for describing a foot-portion inclination angle measured by the measurement device 10. The foot-portion inclination angle represents an angle of a planta with respect to the ground (an XY plane). For the foot-portion inclination angle, a state in which a toe faces upward (dorsiflexion) is defined as negative. For the foot-portion inclination angle, a state in which the toe faces downward (plantarflexion) is defined as positive. For example, there may be cases where positive and negative signs for a foot-portion inclination angle may be defined in a reversed manner. In such a case, dorsiflexion is defined as positive and plantarflexion is defined as negative.

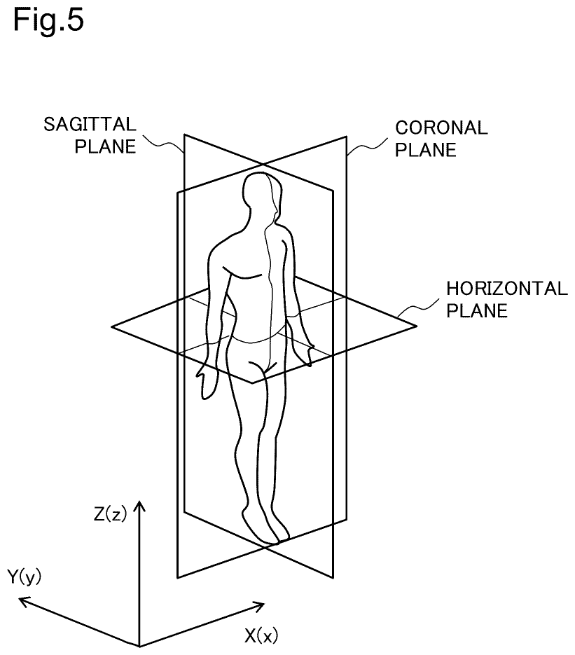

FIG. 5 is a conceptual diagram for describing surfaces set for a human body (also referred to as human body surfaces). One of the human body surfaces, which divides the body into left and right, is referred to as a sagittal plane. One of the human body surfaces, which divides the body into front and back, is referred to as a coronal plane. One of the human body surfaces, which divides the body horizontally, is referred to as a horizontal plane. In a state where a center line of the foot is standing up and faces the front in the one of the travel directions, as illustrated in FIG. 5, the world coordinate system coincides with the local coordinate system. Rotation in the sagittal plane around the X axis serving as a rotation axis is defined as roll. Rotation in the coronal plane around the Y axis serving as the rotation axis is defined as pitch. Rotation in the horizontal plane around the Z axis serving as the rotation axis is defined as yaw. A rotation angle in the sagittal plane around the X axis serving as the rotation axis is referred to as a roll angle. A rotation angle in the coronal plane around the Y axis serving as the rotation axis is referred to as a pitch angle. A rotation angle in the horizontal plane around the Z axis serving as the rotation axis is referred to as a yaw angle. A foot-portion inclination angle and a roll angle will be hereinafter used synonymously.

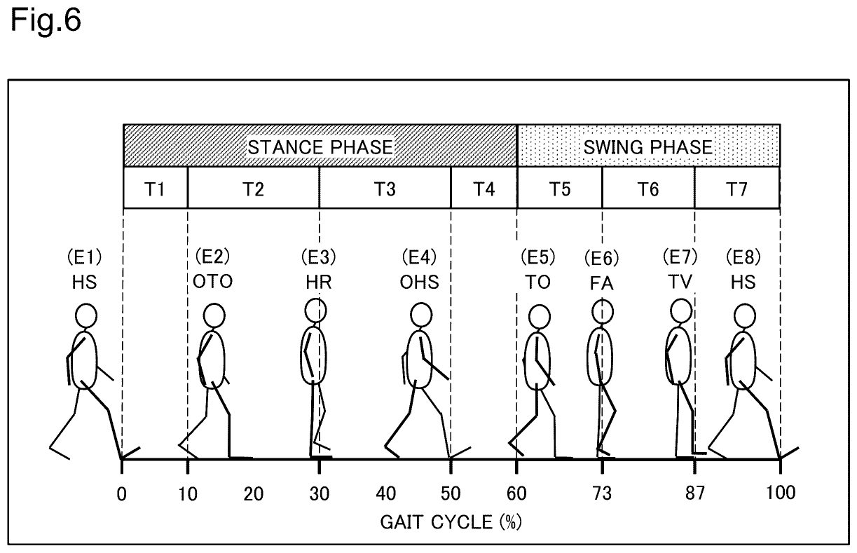

FIG. 6 is a conceptual diagram for describing one gait cycle based on the right foot. One gait cycle based on the left foot is also similar or identical to that of the right foot. A horizontal axis illustrated in FIG. 6 indicates one gait cycle of the right foot where a time point at which a heel of the right foot lands on the ground serves as a starting point and a time point at which the heel of the right foot next lands on the ground serves as an ending point. The horizontal axis illustrated in FIG. 6 is normalized by regarding one gait cycle as 100%. One gait cycle of one foot is roughly divided into a stance phase in which at least a part on the back side of the foot is in contact with the ground and a swing phase in which the back side of the foot is separated from the ground. The horizontal axis illustrated in FIG. 6 is normalized to allow the stance phase to be 60% and the swing phase to be 40%. The stance phase is further subdivided into a load response period T1, a mid-stance period T2, a terminal stance period T3, and a pre-swing period T4. The swing phase is further subdivided into an initial swing period T5, a mid-swing period T6, and a terminal swing period T7. FIG. 6 is a mere example, and there is no intention of limiting periods each forming one gait cycle and names of these periods, for example.

As illustrated in FIG. 6, a plurality of gait events occur during a gate. E1 represents a phenomenon in which the heel of the right foot is in contact with the ground (heel strike) (heel contact: HC). E2 represents a phenomenon in which a toe of the left foot is separated from the ground (separation of toe of opposite foot from ground) in a state where the sole of the right foot is in contact with the ground (opposite toe off: OTO). E3 represents a phenomenon in which the heel of the right foot rises (heel rising) in a state where the sole of the right foot is in contact with the ground (heel rise: HR). E4 represents a phenomenon in which the heel of the left foot is in contact with the ground (heel rising) (opposite heel strike: OHS). E5 represents a phenomenon in which the toe of the right foot is separated from the ground (separation of toe from ground) in a state where the sole of the left foot is in contact with the ground (toe off: TO). E6 represents a phenomenon in which the left foot and the right foot are crossing (crossing feet) in a state where the sole of the left foot is in contact with the ground (foot adjacent: FA). E7 represents a phenomenon in which a tibia of the right foot is approximately vertical to the ground (vertical tibia) in a state where the sole of the left foot is in contact with the ground (tibia vertical: TV). E8 represents a phenomenon in which the heel of the right foot is in contact with the ground (heel strike) (heel contact: (HC)). E8 corresponds to the ending point of the gait cycle starting from E1 and corresponds to the starting point of a next gait cycle. FIG. 6 is a mere example, and there is no intention of limiting phenomena that occur during gaits or names of these phenomena. The detection system 1 according to the present example embodiment detects heel strike as a gait event.

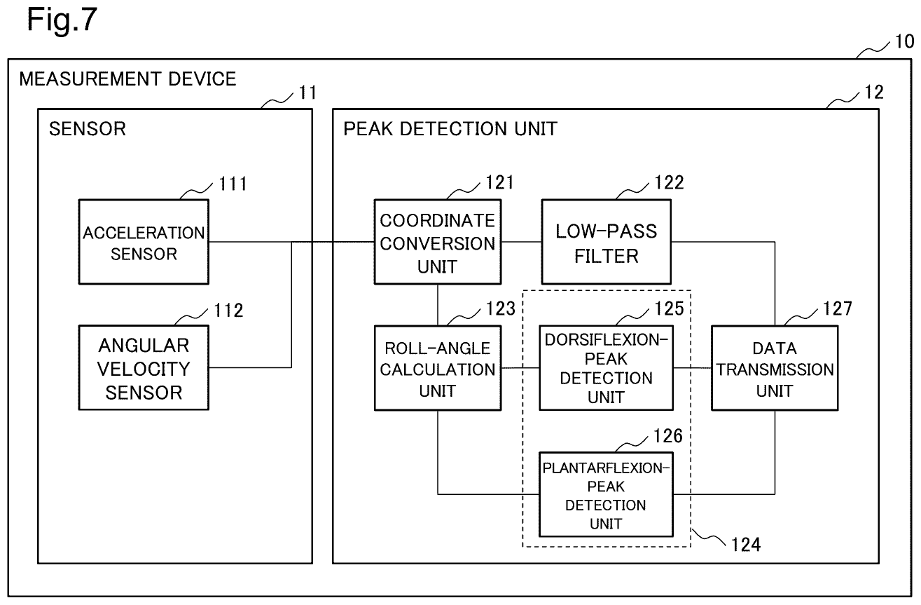

FIG. 7 is a block diagram illustrating an example of a configuration of the measurement device 10. The measurement device 10 includes the sensor 11 and a peak detection unit 12. The sensor 11 includes an acceleration sensor 111 and an angular velocity sensor 112. The peak detection unit 12 includes a coordinate conversion unit 121, a low-pass filter 122, a roll-angle calculation unit 123, a dorsiflexion-peak detection unit 125, a plantarflexion-peak detection unit 126, and a data transmission unit 127. The dorsiflexion-peak detection unit 125 and the plantarflexion-peak detection unit 126 form a plantar-angle-peak detection unit 124.

The acceleration sensor 111 is a sensor that measures degrees of acceleration (also referred to as spatial acceleration) in the three axial directions. The acceleration sensor 111 measures degrees of acceleration in the three axial directions as physical quantities about a movement of the foot. The acceleration sensor 111 outputs the measured degrees of acceleration in the three axial directions to the peak detection unit 12. For example, it is possible to use a sensor of a piezoelectric type, a piezo-resistive type, or a capacitance type as the acceleration sensor 111. For a sensor used as the acceleration sensor 111, there is no added limitation in its measurement method as long as it is possible to measure acceleration.

The angular velocity sensor 112 is a sensor that measures an angular velocity (also referred to as a spatial angular velocity) around each of the three axes. The angular velocity sensor 112 measures an angular velocity around each of the three axes as physical quantities about a movement of the foot. The angular velocity sensor 112 outputs the measured angular velocity to the peak detection unit 12. For example, it is possible to use a sensor of a vibration type or a capacitance type as the angular velocity sensor 112. For a sensor used as the angular velocity sensor 112, there is no added limitation in its measurement method as long it is possible to measure an angular velocity.

The sensor 11 is achieved by, for example, an inertial measurement device that measures acceleration and an angular velocity. An inertial measurement unit (IMU) is an example of the inertial measurement device. The IMU includes the acceleration sensor 111 that measures degrees of acceleration in the three axial directions and the angular velocity sensor 112 that measures an angular velocity around each of the three axes. The sensor 11 may be achieved by an inertial measurement device adopting vertical gyro (VG) or attitude heading (AHRS), for example. The sensor 11 may be achieved based on a global positioning system/inertial navigation system (GPS/INS). The sensor 11 may be achieved by a device other than an inertial measurement device as long as it is possible to measure a physical quantity about a movement of the foot. The sensor 11 may include a sensor other than the acceleration sensor 111 and the angular velocity sensor 112. Description of other sensors that may be included in the sensor 11 is omitted.

The coordinate conversion unit 121 acquires acceleration data and angular velocity data from the sensor 11. The coordinate conversion unit 121 calculates a quaternion and travel direction acceleration using the acceleration data and the angular velocity data. The coordinate conversion unit 121 calculates a quaternion representing an attitude of the sensor 11 using an algorithm called Madgwick Filter. The coordinate conversion unit 121 calculates travel direction acceleration that has been converted in coordinate system from the local coordinate system of the sensor 11 to the world coordinate system. The coordinate conversion unit 121 outputs the calculated quaternion and travel direction acceleration.

The low-pass filter 122 acquires the travel direction acceleration from the coordinate conversion unit 121. The low-pass filter 122 removes a high-frequency component in the travel direction acceleration to perform smoothing. The low-pass filter 122 outputs the travel direction acceleration having undergone smoothing (also referred to as the smoothed travel direction acceleration). Smoothed travel direction acceleration will also be hereinafter referred to as travel direction acceleration.

The roll-angle calculation unit 123 acquires the quaternion from the coordinate conversion unit 121. The roll-angle calculation unit 123 calculates an Euler angle indicating the attitude of the sensor 11 using the quaternion. The Euler angle calculated by the roll-angle calculation unit 123 represents a foot-portion inclination angle (a roll angle). The roll-angle calculation unit 123 outputs the calculated roll angle.

The dorsiflexion-peak detection unit 125 acquires the roll angle from the roll-angle calculation unit 123. The dorsiflexion-peak detection unit 125 detects a time at which the foot portion dorsi-flexes at a highest degree in one gait cycle (also referred to as a dorsiflexion peak time) from time-series data of the acquired roll angle. For example, the dorsiflexion-peak detection unit 125 detects a time at which the time-series data of the roll angle takes a minimum value as a dorsiflexion peak time. For example, the dorsiflexion-peak detection unit 125 detects, as a dorsiflexion peak time, a time at which the roll angle takes a minimum value in the time-series data of the roll angle and the minimum value falls below a threshold value. The dorsiflexion-peak detection unit 125 outputs the detected dorsiflexion peak time.

The plantarflexion-peak detection unit 126 acquires the roll angle from the roll-angle calculation unit 123. The plantarflexion-peak detection unit 126 detects a time at which the foot portion plantar-flexes at a highest degree in one gait cycle (also referred to as a plantarflexion peak time) from the time-series data of the acquired roll angle. For example, the plantarflexion-peak detection unit 126 detects a time at which the time-series data of the roll angle takes a maximum value as a plantarflexion peak time. For example, the plantarflexion-peak detection unit 126 detects, as a plantarflexion peak time, a time at which the roll angle takes a maximum value in the time-series data of the roll angle and the maximum value exceeds the threshold value. The plantarflexion-peak detection unit 126 outputs the detected plantarflexion peak time.

The dorsiflexion-peak detection unit 125 and the plantarflexion-peak detection unit 126 may form a single configuration (the plantar-angle-peak detection unit 124). For example, the plantar-angle-peak detection unit 124 detects a time at which time-series data of a roll angle takes a minimum value as a dorsiflexion peak time. For example, the plantar-angle-peak detection unit 124 detects a time at which the time-series data of the roll angle takes a maximum value as a plantarflexion peak time. The plantar-angle-peak detection unit 124 sequentially outputs dorsiflexion peak times and plantarflexion peak times alternately detected.

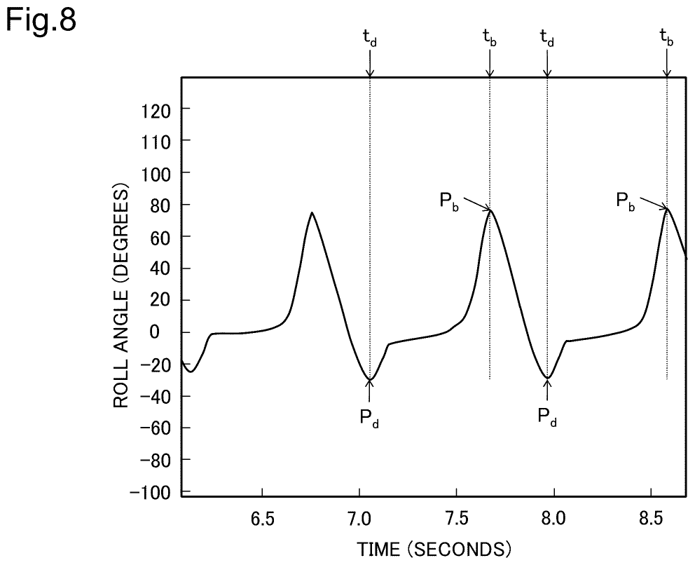

FIG. 8 is an example of time-series data of a roll angle. A time of a minimum point P_{d} at which the time-series data of the roll angle takes a minimum value represents a dorsiflexion peak time t_{d}. A time of a maximum point P_{b} at which the time-series data of the roll angle takes a maximum value represents a plantarflexion peak time t_{b}. For example, there may be cases where positive and negative signs for a foot-portion inclination angle may be defined in a reversed manner. In such a case, a time of a minimum point of time-series data of a roll angle corresponds to a plantarflexion peak time, and a time of a maximum point of the time-series data of the roll angle corresponds to a dorsiflexion peak time.

The data transmission unit 127 acquires the travel direction acceleration from the low-pass filter 122. The data transmission unit 127 acquires the dorsiflexion peak time from the dorsiflexion-peak detection unit 125. The data transmission unit 127 acquires the plantarflexion peak time from the plantarflexion-peak detection unit 126. The data transmission unit 127 transmits transmission data including the travel direction acceleration, the dorsiflexion peak time, and the plantarflexion peak time to the detection device 13. The transmission data may include data such as left-and-right direction acceleration (X-direction acceleration), vertical direction acceleration (Z-direction acceleration), and an angular velocity and an angle around each of the three axes, for example.

### [Detection Device]

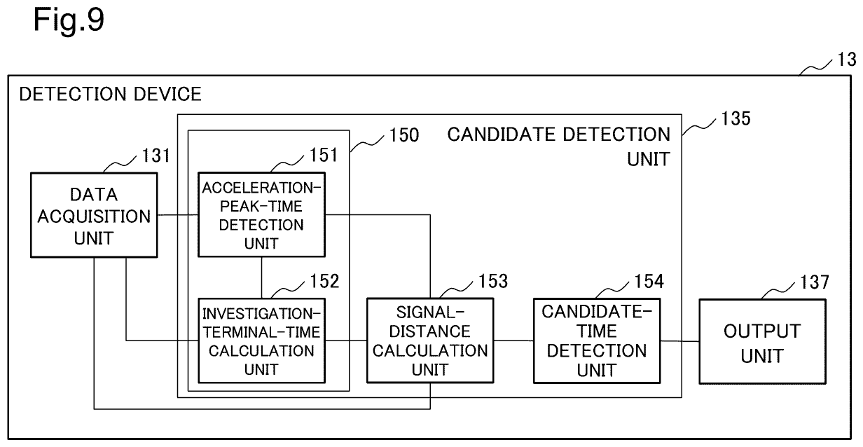

FIG. 9 is a block diagram illustrating an example of a configuration of the detection device 13. The detection device 13 includes a data acquisition unit 131, a candidate detection unit 135, and an output unit 137. The candidate detection unit 135 includes an acceleration-peak-time detection unit 151, an investigation-terminal-time calculation unit 152, a signal-distance calculation unit 153, and a candidate-time detection unit 154. The acceleration-peak-time detection unit 151 and the investigation-terminal-time calculation unit 152 form an investigation-time-period setting unit 150.

The data acquisition unit 131 acquires the transmission data from the measurement device 10. The data acquisition unit 131 outputs the travel direction acceleration and the dorsiflexion peak time included in the acquired transmission data to the acceleration-peak-time detection unit 151. The travel direction acceleration includes time-series data of values at signal points at measurement timings (times) for the sensor data. The data acquisition unit 131 outputs the dorsiflexion peak time and the plantarflexion peak time included in the acquired transmission data to the investigation-terminal-time calculation unit 152.

For example, the data acquisition unit 131 receives the transmission data from the measurement device 10 via wireless communication. For example, the data acquisition unit 131 is configured to receive the transmission data from the measurement device 10 via a wireless communication function (not illustrated) conforming to a standard such as Bluetooth (registered trademark) or WiFi (registered trademark). The communication function of the data acquisition unit 131 may conform to a standard other than Bluetooth (registered trademark) or WiFi (registered trademark). The data acquisition unit 131 may receive transmission data from the measurement device 10 via a wire such as a cable. The data acquisition unit 131 may receive transmission data via a communication function of a mobile terminal, for example, on which the detection device 13 is mounted.

The acceleration-peak-time detection unit 151 acquires the travel direction acceleration and the dorsiflexion peak time from the data acquisition unit 131. The acceleration-peak-time detection unit 151 detects a time (also referred to as an acceleration peak time) at which the travel direction acceleration takes a maximum value in an investigation time period before and after the dorsiflexion peak time. That is, the acceleration-peak-time detection unit 151 detects the acceleration peak time based on the dorsiflexion peak time. The acceleration peak time represents a starting time of an investigation time period (also referred to as a first investigation time period) for a heel strike time. When the positive and negative signs of the travel direction acceleration are reversed, a time at which the travel direction acceleration takes a minimum value corresponds to an acceleration peak time. For example, the acceleration-peak-time detection unit 151 detects an acceleration peak time within a predetermined time period based on a dorsiflexion peak time. For example, the acceleration-peak-time detection unit 151 detects an acceleration peak time in a data range corresponding to several samples based on a dorsiflexion peak time. The acceleration-peak-time detection unit 151 outputs the detected acceleration peak time to the signal-distance calculation unit 153.

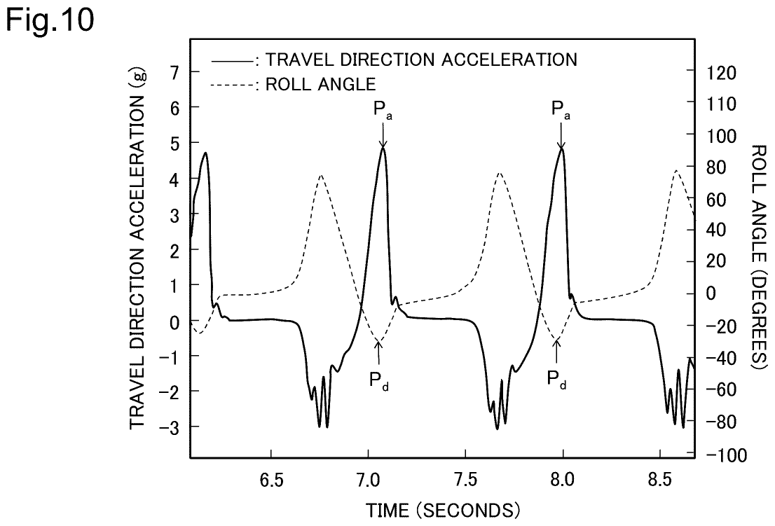

FIG. 10 is a conceptual diagram for describing an example of acceleration peaks detected by the acceleration-peak-time detection unit 151. FIG. 10 illustrates an example of a waveform of time-series data of the travel direction of the foot portion, which has been measured in accordance with gaits of the user, and a waveform of time-series data of an angle (a roll angle) in the sagittal plane. The acceleration-peak-time detection unit 151 detects an acceleration peak time in an investigation time period before and after a dorsiflexion peak time t_{d}. In a normal gait, a timing at which time-series data of a roll angle takes a minimum value (a dorsiflexion peak) appears around heel strike. A maximum peak appearing in time-series data of travel direction acceleration is derived due to sudden deceleration that occurs immediately before heel strike. A difference between a dorsiflexion peak time and an acceleration peak time normally falls within a range of approximately 10% of a gait cycle. An investigation time period for an acceleration peak may be set to a time range of approximately 10% of a gait cycle.

The investigation-terminal-time calculation unit 152 acquires the dorsiflexion peak time and the plantarflexion peak time from the data acquisition unit 131. The investigation-terminal-time calculation unit 152 detects, as an investigation terminal time, a time at a midpoint between the dorsiflexion peak time and the plantarflexion peak time (also referred to as a time in a mid-stance period). An investigation terminal time (also referred to as a first investigation terminal time) calculated by the investigation-terminal-time calculation unit 152 represents a time at which the first investigation time period ends. The investigation-terminal-time calculation unit 152 outputs the detected first investigation terminal time to the signal-distance calculation unit 153.

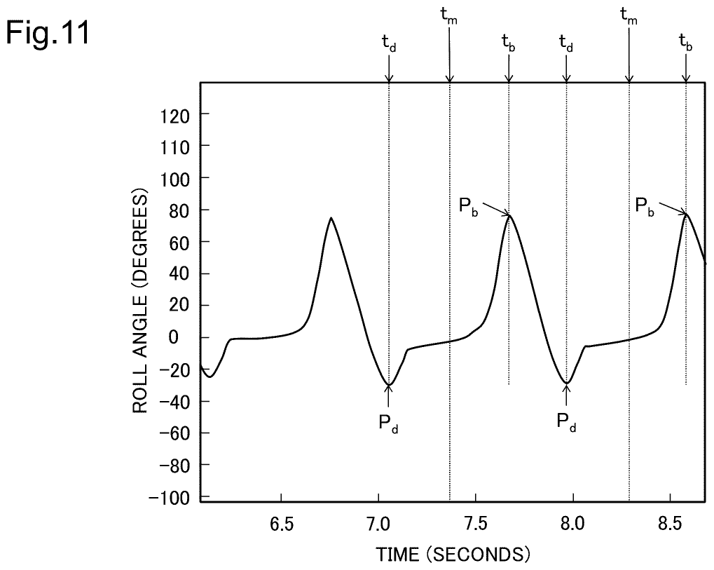

FIG. 11 is an example of time-series data of a roll angle. A midpoint between a dorsiflexion peak time t_{d} and a plantarflexion peak time to represents a time in a mid-stance period tₘ. A time period between times in two consecutive mid-stance periods tₘ corresponds to one gait cycle.

The signal-distance calculation unit 153 acquires the travel direction acceleration from the data acquisition unit 131. The signal-distance calculation unit 153 may acquire travel direction acceleration from the acceleration-peak-time detection unit 151. The signal-distance calculation unit 153 acquires the acceleration peak time from the acceleration-peak-time detection unit 151. The signal-distance calculation unit 153 further acquires the first investigation terminal time from the investigation-terminal-time calculation unit 152. The signal-distance calculation unit 153 sets a time period from the acceleration peak time to the first investigation terminal time as an investigation time period (also referred to as a first investigation time period).

The signal-distance calculation unit 153 draws a straight line passing through signal points at the acceleration peak time and the first investigation terminal time in the waveform of the time-series data of the travel direction acceleration (also referred to as a first reference straight line). A waveform of time-series data of travel direction acceleration is expressed in a graph in which a horizontal axis indicates time and a vertical axis indicates travel direction acceleration. For example, the signal-distance calculation unit 153 draws a first reference straight line passing through signal points at an acceleration peak time and a first investigation terminal time in a waveform of time-series data of travel direction acceleration. For example, the signal-distance calculation unit 153 may draw, as a first reference straight line, a line segment coupling signal points of travel direction acceleration at an acceleration peak time and a first investigation terminal time.

In the first investigation time period, the signal-distance calculation unit 153 calculates, as a first signal distance, a Euclidean distance between a signal point at each time in the waveform of the time-series data of the travel direction acceleration and the first reference straight line. A first signal distance corresponds to a length of a perpendicular line drawn from a signal point at each time of travel direction acceleration to a first reference straight line. The signal-distance calculation unit 153 outputs the first signal distance calculated for each time in the waveform of the time-series data of the travel direction acceleration to the candidate-time detection unit 154.

The candidate-time detection unit 154 acquires, from the signal-distance calculation unit 153, the first signal distance calculated for each time in the waveform of the time-series data of the travel direction acceleration. The candidate-time detection unit 154 detects a signal point at which the first signal distance reaches maximum. A signal point at which a first signal distance reaches maximum is referred to as a feature signal point. The candidate-time detection unit 154 detects a time of the feature signal point as a candidate time for heel strike. The candidate-time detection unit 154 outputs the detected candidate time (also referred to as a first candidate time) to the output unit 137.

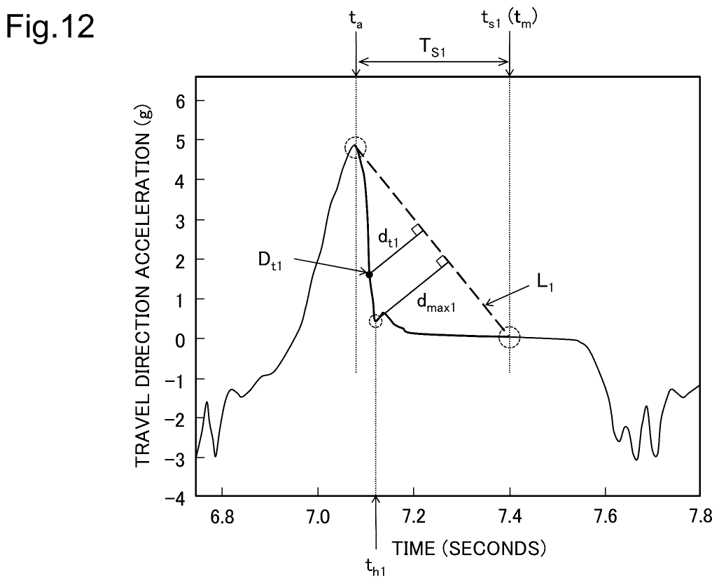

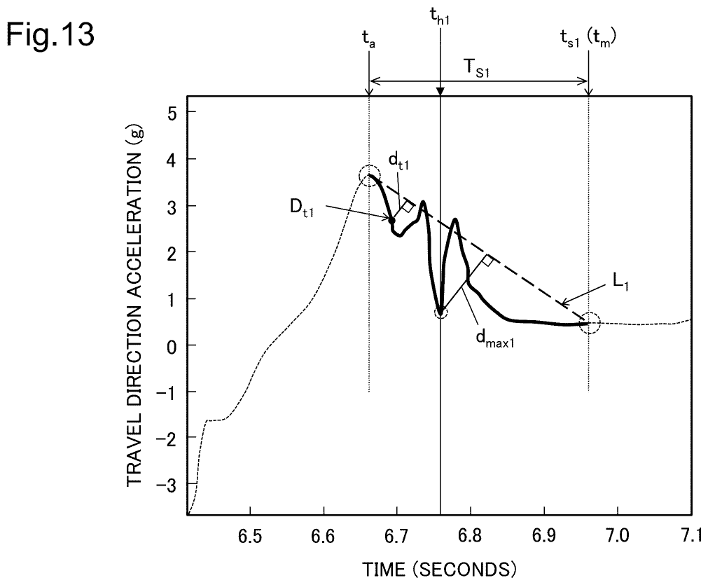

FIGS. 12 and 13 are graphs for describing examples of detecting a first candidate time tₕ₁ from a waveform of time-series data of travel direction acceleration. The signal-distance calculation unit 153 draws a first reference straight line L₁ passing through signal points at an acceleration peak time tₐ and a first investigation terminal time tₛ₁ in a waveform of time-series data of travel direction acceleration. The first investigation terminal time tₛ₁ corresponds to a time in a mid-stance period tₘ. The signal-distance calculation unit 153 calculates a Euclidean distance between a signal point Du at each time in the waveform of the time-series data of the travel direction acceleration and the first reference straight line L₁ in a first investigation time period Tₛ₁ between the acceleration peak time tₐ and the first investigation terminal time tₛ₁. The calculated Euclidean distance represents a first signal distance dₜ₁. The candidate-time detection unit 154 detects, as a first candidate time tₕ₁, a time at which the first signal distance dₜ₁ reaches maximum (dₘₐₓ₁) in the first investigation time period Tₛ₁.

The output unit 137 acquires the first candidate time from the candidate-time detection unit 154. The output unit 137 outputs the acquired first candidate time as a heel strike time. For example, the output unit 137 outputs a heel strike time to a non-illustrated system or device. For example, the output unit 137 outputs a heel strike time to another piece of software installed inside a terminal device on which the detection device 13 has been mounted. For example, the output unit 137 outputs a heel strike time from the terminal device on which the detection device 13 has been mounted to a non-illustrated system or device executed in a cloud or a server. There is no limitation in destination of outputting a heel strike time.

For example, the detection device 13 is coupled to an external system constructed in a cloud or a server via a mobile terminal (not illustrated) carried by the user. A mobile terminal is a portable terminal device having a communication function. For example, the mobile terminal is a portable communication device having a communication function, such as a smartphone, a smart watch, or a mobile phone. For example, the detection device 13 is coupled to a mobile terminal via a wire such as a cable. For example, the detection device 13 is coupled to a mobile terminal via wireless communication. For example, the detection device 13 is coupled to a mobile terminal via a wireless communication function (not illustrated) conforming to a standard such as Bluetooth (registered trademark) or WiFi (registered trademark). The communication function of the detection device 13 may conform to a standard other than Bluetooth (registered trademark) or WiFi (registered trademark). A heel strike time may be used by an application installed in a mobile terminal. In such a case, the mobile terminal executes processing using the heel strike time with application software installed in the mobile terminal, for example.

### (Operation)

Next, operation of the detection device 13 included in the detection system 1 will now be described herein with reference to the accompanying drawings. FIG. 13 is a flowchart for describing an example of operation of the detection device 13. In the description with reference to the flowchart illustrated in FIG. 13, the detection device 13 will be described as a main operational body.

In FIG. 13, the detection device 13 first acquires transmission data transmitted from the measurement device 10 (step S11). The transmission data includes travel direction acceleration, a dorsiflexion peak time, and a plantarflexion peak time.

Next, the detection device 13 detects an acceleration peak time based on the dorsiflexion peak time in a waveform of time-series data of the travel direction acceleration (step S12). For example, the detection device 13 detects an acceleration peak time within a time range of approximately 10% of a gait cycle based on a dorsiflexion peak time. For example, the detection device 13 detects an acceleration peak time within a data range corresponding to several samples based on a dorsiflexion peak time.

Next, the detection device 13 calculates, as a first investigation terminal time, a time at a midpoint between the dorsiflexion peak time and the plantarflexion peak time (a time in a mid-stance period) (step S13). The first investigation terminal time corresponds to a time in a mid-stance period.

Next, the detection device 13 calculates a first signal distance in a first investigation time period between the acceleration peak time and the first investigation terminal time (step S14). For example, the detection device 13 draws a first reference straight line passing through signal points at the acceleration peak time and the first investigation terminal in the waveform of the time-series data of the travel direction acceleration. In the investigation time period, the detection device 13 calculates a Euclidean distance between a signal point at each time in the waveform of the time-series data of the travel direction acceleration and the first reference straight line (a first signal distance).

Next, the detection device 13 detects, as a first candidate time, a time at which the first signal distance reaches maximum in the first investigation time period (step S15).

Next, the detection device 13 outputs the detected first candidate time as a heel strike time (step S16). The heel strike time outputted from the detection device 13 is used for detecting a gait event and estimating a physical condition of the user, for example.

As described above, the detection system according to the present example embodiment includes the measurement device and the detection device. The measurement device includes the sensor and the peak detection unit. The sensor is installed on footwear of the user. The sensor measures spatial acceleration and a spatial angular velocity. The sensor generates sensor data regarding a movement of the foot using the measured spatial acceleration and spatial angular velocity. The sensor outputs the generated sensor data, and the peak detection unit acquires time-series data of the sensor data. The peak detection unit smooths time-series data of travel direction acceleration, which is included in the sensor data. The peak detection unit detects a dorsiflexion peak time and a plantarflexion peak time from time-series data of a roll angle, which is included in the sensor data. The peak detection unit outputs data including the smoothed travel direction acceleration, the dorsiflexion peak time, and the plantarflexion peak time to the detection device.

The detection device includes the data acquisition unit, the candidate detection unit, and the output unit. The data acquisition unit acquires data including a dorsiflexion peak time, a plantarflexion peak time, and travel direction acceleration, which are acquired from sensor data regarding a movement of the foot. The candidate detection unit calculates, as a first investigation terminal time, a time in a mid-stance period, which corresponds to a time at a midpoint between the dorsiflexion peak time and the plantarflexion peak time. The candidate detection unit sets a time period from the acceleration peak time to the first investigation terminal time as a first investigation time period. The candidate detection unit sets a first reference straight line passing through a signal point of the travel direction acceleration at the acceleration peak time and a signal point of the travel direction acceleration at the first investigation terminal time. The candidate detection unit calculates a first signal distance corresponding to a Euclidean distance of the signal point of the travel direction acceleration with respect to the first reference straight line for a signal point of the travel direction acceleration, which is included in the first investigation time period. The candidate detection unit detects a time of a feature signal point at which the calculated first signal distance takes a maximum value as a candidate time. The output unit outputs the detected candidate time as a heel strike time.

In the present example embodiment, a first investigation time period starting from an acceleration peak time acquired from sensor data measured by the sensor installed on a foot portion of the user and ending at a first investigation terminal time is set. In the present example embodiment, a time of a feature signal point at which a first signal distance corresponding to a Euclidean distance of a signal point of travel direction acceleration with respect to a first reference straight line set in a first investigation time period takes a maximum value is detected as a candidate time. In the present example embodiment, the candidate time detected in the set first investigation time period is detected as a heel strike time. According to the present example embodiment, it is possible to uniquely detect heel strike in a gait of the user using data measured by the sensor installed on the foot portion of the user.

It is possible to apply the method according to the present example embodiment to gait analysis in fields such as medical care and healthcare. Heel grounding is an important gait event in gait analysis. For example, a heel strike time detected with the method according to the present example embodiment is used for analyzing a relationship between an angle of a foot portion at the heel strike time and a certain disease. For example, a heel strike time detected with the method according to the present example embodiment is used as a reference when another gait event is to be detected.

### Second Example Embodiment

Next, a detection device according to a second example embodiment will now be described herein with reference to the accompanying drawings. The detection device according to the present example embodiment is different from that according to the first example embodiment in an investigation time period for a candidate time for a heel strike time. The detection device according to the present example embodiment acquires transmission data from the measurement device according to the first example embodiment.

### (Configuration)

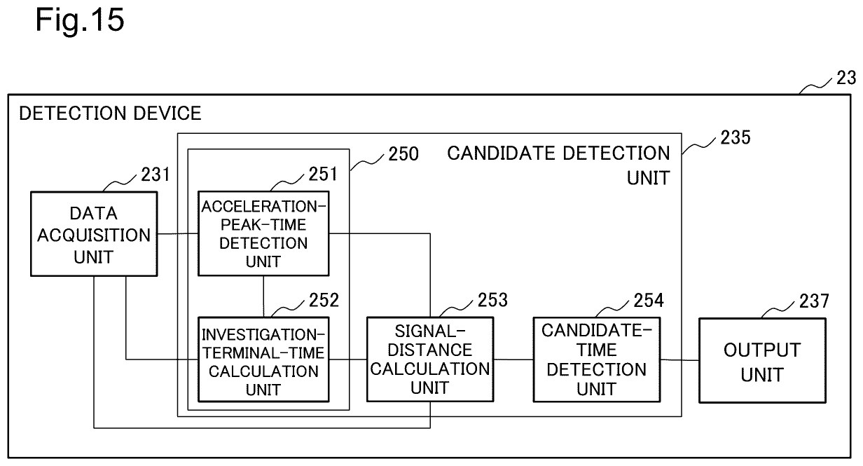

FIG. 15 is a block diagram illustrating an example of a configuration of a detection device 23. The detection device 23 includes a data acquisition unit 231, a candidate detection unit 235, and an output unit 237. The candidate detection unit 235 includes an acceleration-peak-time detection unit 251, an investigation-terminal-time calculation unit 252, a signal-distance calculation unit 253, and a candidate-time detection unit 254. The acceleration-peak-time detection unit 251 and the investigation-terminal-time calculation unit 252 form an investigation-time-period setting unit 250.

The data acquisition unit 231 has a similar or identical configuration to that of the data acquisition unit 131 according to the first example embodiment. The data acquisition unit 231 acquires transmission data from the measurement device (not illustrated). The data acquisition unit 231 outputs travel direction acceleration and a dorsiflexion peak time included in the transmission data to the acceleration-peak-time detection unit 251. The travel direction acceleration includes time-series data of values at signal points at measurement timings (times) for the sensor data. The data acquisition unit 231 outputs the dorsiflexion peak time and a plantarflexion peak time included in the transmission data to the investigation-terminal-time calculation unit 252.

The acceleration-peak-time detection unit 251 has a similar or identical configuration to that of the acceleration-peak-time detection unit 151 included in the detection device 13 according to the first example embodiment. The acceleration-peak-time detection unit 251 acquires the travel direction acceleration and the dorsiflexion peak time from the data acquisition unit 231. The acceleration-peak-time detection unit 251 detects an acceleration peak time at which the travel direction acceleration takes a maximum value in an investigation time period before and after the dorsiflexion peak time. That is, the acceleration-peak-time detection unit 251 detects the acceleration peak time based on the dorsiflexion peak time. The acceleration peak time represents a starting time of an investigation time period for a heel strike time (also referred to as a second investigation time period). When the positive and negative signs of the travel direction acceleration are reversed, a time at which the travel direction acceleration takes a minimum value corresponds to an acceleration peak time. The acceleration-peak-time detection unit 251 outputs the detected acceleration peak time to the signal-distance calculation unit 253.

The investigation-terminal-time calculation unit 252 acquires the dorsiflexion peak time and the plantarflexion peak time from the data acquisition unit 231. The investigation-terminal-time calculation unit 252 acquires the acceleration peak time from the acceleration-peak-time detection unit 251. The investigation-terminal-time calculation unit 252 detects a time at a midpoint between the dorsiflexion peak time and the plantarflexion peak time (a time in a mid-stance period). The investigation-terminal-time calculation unit 252 detects, as one gait cycle, a time period between the times in the consecutive mid-stance periods. For example, the investigation-terminal-time calculation unit 252 calculates, as one gait cycle, a difference between a time in a latest mid-stance period, which is being verified, and a time in a previous mid-stance period, which is immediately before the time in the latest mid-stance period. The investigation-terminal-time calculation unit 252 calculates, as an investigation terminal time, a time after a predetermined ratio of the one gait cycle from the acceleration peak time. In an ordinary gait, heel strike occurs in a time period of approximately 10% immediately after travel direction acceleration reaches a peak. The predetermined ratio of one gait cycle may be set to approximately 10% of one gait cycle. An investigation terminal time (also referred to as a second investigation terminal time) calculated by the investigation-terminal-time calculation unit 252 represents a time at which the second investigation time period ends. The investigation-terminal-time calculation unit 252 outputs the calculated second investigation terminal time to the signal-distance calculation unit 253.

The signal-distance calculation unit 253 acquires the travel direction acceleration from the data acquisition unit 231. The signal-distance calculation unit 253 may acquire the travel direction acceleration from the acceleration-peak-time detection unit 251. The signal-distance calculation unit 253 acquires the acceleration peak time from the acceleration-peak-time detection unit 251. The signal-distance calculation unit 253 further acquires the second investigation terminal time from the investigation-terminal-time calculation unit 252. The signal-distance calculation unit 253 sets a time period from the acceleration peak time to the second investigation terminal time as an investigation time period (also referred to as a second investigation time period).

The signal-distance calculation unit 253 draws a straight line passing through signal points at the acceleration peak time and the second investigation terminal time in a waveform of time-series data of the travel direction acceleration (a second reference straight line). A waveform of time-series data of travel direction acceleration is expressed in a graph in which a horizontal axis indicates time and a vertical axis indicates travel direction acceleration. For example, the signal-distance calculation unit 253 draws a second reference straight line passing through signal points at an acceleration peak time and a second investigation terminal time in a waveform of time-series data of travel direction acceleration. For example, the signal-distance calculation unit 253 may draw, as a second reference straight line, a line segment coupling signal points of travel direction acceleration at an acceleration peak time and a second investigation terminal time.

In the second investigation time period, the signal-distance calculation unit 253 calculates, as a second signal distance, a Euclidean distance between a signal point at each time in the waveform of the time-series data of the travel direction acceleration and the second reference straight line. A second signal distance corresponds to a length of a perpendicular line drawn from a signal point at each time of travel direction acceleration to a second reference straight line. The signal-distance calculation unit 253 outputs the second signal distance calculated for each time of the waveform of the time-series data of the travel direction acceleration to the candidate-time detection unit 254.

The candidate-time detection unit 254 acquires, from the signal-distance calculation unit 253, the second signal distance calculated for each time in the waveform of the time-series data of the travel direction acceleration. The candidate-time detection unit 254 detects a signal point at which the second signal distance reaches maximum. A signal point at which a second signal distance reaches maximum is referred to as a feature signal point. The candidate-time detection unit 254 detects a time of the feature signal point as a candidate time for heel strike. The candidate-time detection unit 254 outputs the detected candidate time (also referred to as a second candidate time) to the output unit 237.

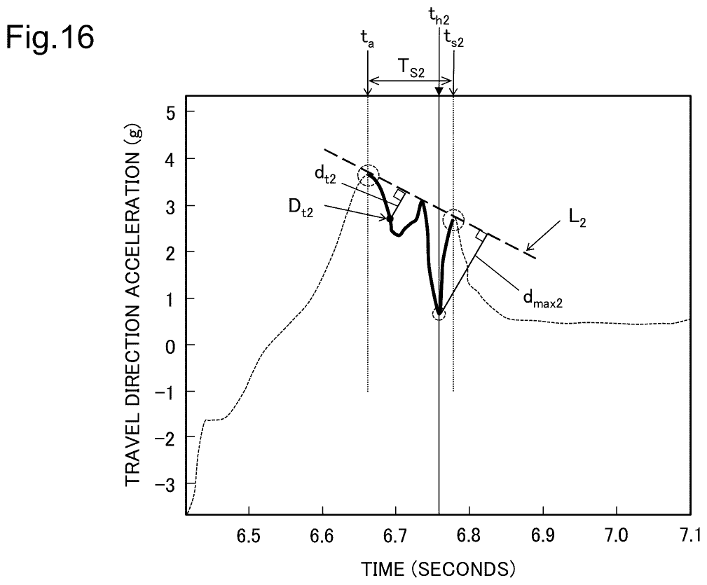

FIG. 16 is a graph for describing an example of detecting a second candidate time tₕ₂ from a waveform of time-series data of travel direction acceleration. The signal-distance calculation unit 253 draws a second reference straight line L₂ passing through signal points at an acceleration peak time tₐ and a second investigation terminal time tₛ₂ in the waveform of the time-series data of the travel direction acceleration. The signal-distance calculation unit 253 calculates a Euclidean distance between a signal point Dₜ₂ at each time in the waveform of the time-series data of the travel direction acceleration and the second reference straight line L₂ in a second investigation time period Tₛ₂ between the acceleration peak time tₐ and the second investigation terminal time tₛ₂. The calculated Euclidean distance represents a second signal distance dₜ₂. The candidate-time detection unit 254 detects, as a second candidate time tₕ₂, a time at which the second signal distance dₜ₂ reaches maximum (dₘₐₓ₂) in the second investigation time period Tₛ₂. The signal point Dₜ₂ at which the second signal distance dₜ₂ reaches maximum (dₘₐₓ₂) is also referred to as a feature signal point.

The output unit 237 acquires the second candidate time from the candidate-time detection unit 254. The output unit 237 outputs the acquired second candidate time as a heel strike time. For example, the output unit 237 outputs a heel strike time to a non-illustrated system or device. For example, the output unit 237 outputs a heel strike time to another piece of software installed inside a terminal device on which the detection device 23 has been mounted. For example, the output unit 237 outputs a heel strike time from a terminal device on which the detection device 23 has been mounted to a non-illustrated system or device executed in a cloud or a server. There is no limitation in destination of outputting a heel strike time.

### (Operation)

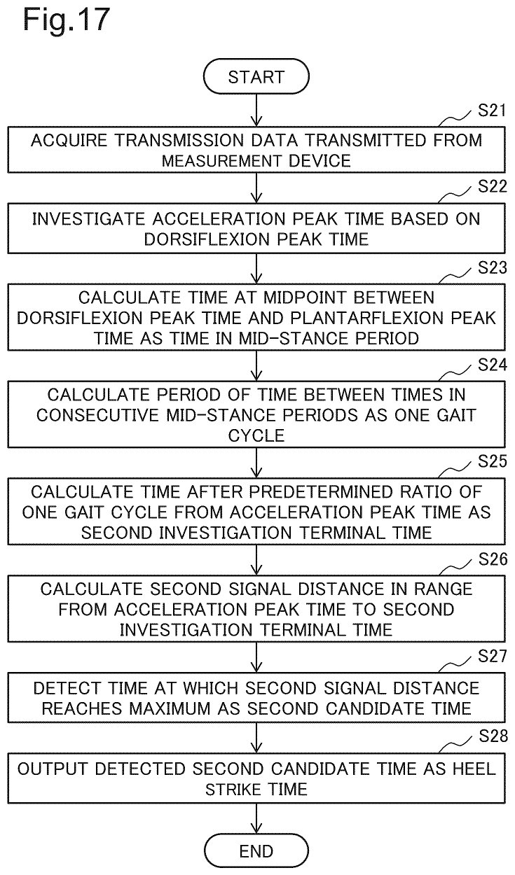

Next, operation of the detection device 23 will now be described herein with reference to the accompanying drawings. FIG. 17 is a flowchart for describing an example of operation of the detection device 23. In the description with reference to the flowchart illustrated in FIG. 17, the detection device 23 will be described as a main operational body.

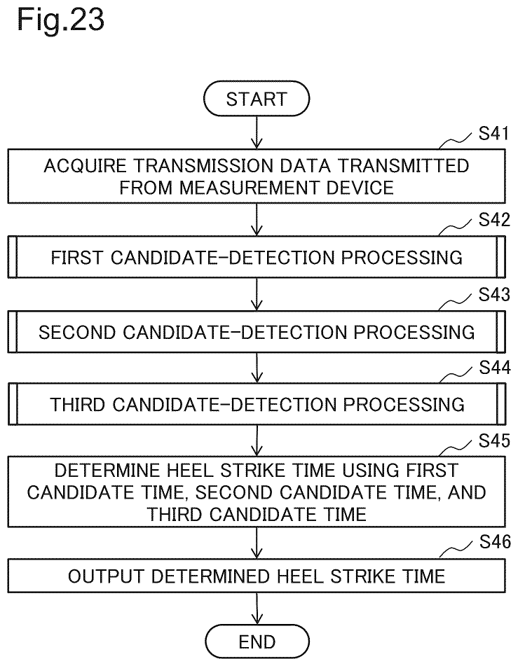

In FIG. 23, the detection device 23 first acquires transmission data transmitted from the measurement device (not illustrated) (step S21). The transmission data includes travel direction acceleration, a dorsiflexion peak time, and a plantarflexion peak time.

Next, the detection device 23 detects an acceleration peak time based on the dorsiflexion peak time in a waveform of time-series data of the travel direction acceleration (step S22). For example, the detection device 23 detects an acceleration peak time within a time range of approximately 10% of a gait cycle based on a dorsiflexion peak time. For example, the detection device 23 detects an acceleration peak time within a data range corresponding to several samples based on a dorsiflexion peak time.

Next, the detection device 23 calculates, as a time in a mid-stance period, a time at a midpoint between the dorsiflexion peak time and the plantarflexion peak time (step S23).

Next, the detection device 23 calculates, as one gait cycle, a period of time between the times in the consecutive mid-stance periods (step S24).

Next, the detection device 23 calculates, as a second investigation terminal time, a time after a predetermined ratio of the one gait cycle from the acceleration peak time (step S25).

Next, the detection device 23 calculates a second signal distance in the second investigation time period between the acceleration peak time and the second investigation terminal time (step S26). For example, the detection device 23 draws a second reference straight line passing through signal points at the acceleration peak time and the second investigation terminal time in the waveform of the time-series data of the travel direction acceleration. In the second investigation time period, the detection device 23 calculates a Euclidean distance between a signal point at each time in the waveform of the time-series data of the travel direction acceleration and the second reference straight line (a second signal distance).

Next, the detection device 23 detects, as a second candidate time, a time at which the second signal distance reaches maximum in the second investigation time period (step S27).

Next, the detection device 23 outputs the detected second candidate time as a heel strike time (step S28). The heel strike time outputted from the detection device 23 is used for detecting a gait event and estimating a physical condition of the user, for example.

As described above, the detection device according to the present example embodiment includes the data acquisition unit, the candidate detection unit, and the output unit. The data acquisition unit acquires data including a dorsiflexion peak time, a plantarflexion peak time, and travel direction acceleration, which are acquired from sensor data regarding a movement of the foot. The candidate detection unit calculates a time in a mid-stance period, which corresponds to a time at a midpoint between the dorsiflexion peak time and the plantarflexion peak time. The candidate detection unit calculates, as one gait cycle, a time period between the times in the consecutive mid-stance periods. The candidate detection unit sets, as a second investigation terminal time, a time after a predetermined ratio of the one gait cycle from the acceleration peak time. The candidate detection unit sets a time period from the acceleration peak time to the second investigation terminal time as a second investigation time period. The candidate detection unit sets a second reference straight line passing through a signal point of the travel direction acceleration at the acceleration peak time and a signal point of the travel direction acceleration at the second investigation terminal time. The candidate detection unit calculates a second signal distance corresponding to a Euclidean distance of the signal point of the travel direction acceleration with respect to the second reference straight line for a signal point of the travel direction acceleration, which is included in the second investigation time period. The candidate detection unit detects a time of a feature signal point at which the calculated second signal distance takes a maximum value as a candidate time. The output unit outputs the detected candidate time as a heel strike time.

In the present example embodiment, a second investigation time period starting from an acceleration peak time acquired from sensor data measured by the sensor installed on a foot portion of the user and ending at a second investigation terminal time is set. In the present example embodiment, a time of a feature signal point at which a second signal distance corresponding to a Euclidean distance of a signal point of travel direction acceleration with respect to a second reference straight line set in a second investigation time period takes a maximum value is detected as a candidate time. In the present example embodiment, the candidate time detected in the second investigation time period is detected as a heel strike time. According to the present example embodiment, it is possible to uniquely detect heel strike in a gait of the user using data measured by the sensor installed on the foot portion of the user.

### Third Example Embodiment

Next, a detection device according to a third example embodiment will now be described herein with reference to the accompanying drawings. The detection device according to the present example embodiment is different from those according to the first and second example embodiments in an investigation time period for a candidate time for a heel strike time. The detection device according to the present example embodiment is further different from those according to the first and second example embodiments in that no reference straight line is used. The detection device according to the present example embodiment acquires transmission data from the measurement device according to the first example embodiment.

### (Configuration)

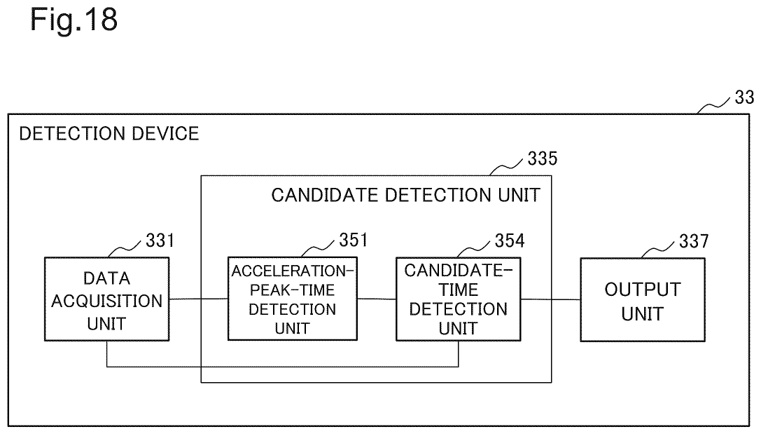

FIG. 18 is a block diagram illustrating an example of a configuration of a detection device 33. The detection device 33 includes a data acquisition unit 331, a candidate detection unit 335, and an output unit 337. The candidate detection unit 335 includes an acceleration-peak-time detection unit 351 and a candidate-time detection unit 354.

The data acquisition unit 331 has a similar or identical configuration to that of the data acquisition unit 131 according to the first example embodiment. The data acquisition unit 331 acquires transmission data from the measurement device (not illustrated). The data acquisition unit 331 outputs travel direction acceleration and a dorsiflexion peak time included in the transmission data to the acceleration-peak-time detection unit 351. The travel direction acceleration includes time-series data of values at signal points at measurement timings (times) for the sensor data. The data acquisition unit 331 outputs the travel direction acceleration included in the transmission data to the candidate-time detection unit 354.

The acceleration-peak-time detection unit 351 has a similar or identical configuration to that of the acceleration-peak-time detection unit 151 included in the detection device 13 according to the first example embodiment. The acceleration-peak-time detection unit 351 acquires the travel direction acceleration and the dorsiflexion peak time from the data acquisition unit 331. The acceleration-peak-time detection unit 351 detects an acceleration peak time at which the travel direction acceleration takes a maximum value in an investigation time period before and after the dorsiflexion peak time. That is, the acceleration-peak-time detection unit 351 detects the acceleration peak time based on the dorsiflexion peak time. When the positive and negative signs of the travel direction acceleration are reversed, a time at which the travel direction acceleration takes a minimum value corresponds to an acceleration peak time. The acceleration-peak-time detection unit 351 outputs the detected acceleration peak time to the candidate-time detection unit 354.

The candidate-time detection unit 354 acquires the travel direction acceleration from the data acquisition unit 331. The candidate-time detection unit 354 acquires the acceleration peak time from the acceleration-peak-time detection unit 351. The candidate-time detection unit 354 sets a time period subsequent to the acceleration peak time as a third investigation time period.

The candidate-time detection unit 354 detects a signal point at which the travel direction acceleration first takes a minimum value in the third investigation time period. When the positive and negative signs of the travel direction acceleration are reversed, the candidate-time detection unit 354 detects a signal point at which travel direction acceleration first takes a maximum value in a third investigation time period. That is, the candidate-time detection unit 354 detects a signal point at which the travel direction acceleration first takes an extreme value in the third investigation time period. In the third investigation time period, a signal point at which the travel direction acceleration first takes an extreme value is referred to as a feature signal point. The candidate-time detection unit 354 detects a time of the feature signal point as a candidate time for heel strike.

The third investigation time period starts at an acceleration peak time tₐ. The third investigation time period may end after a time at which the travel direction acceleration first takes a minimum value after the acceleration peak time tₐ. For example, the candidate-time detection unit 354 compares signal values at a time tₙ₋₁, a time tₙ, and a time tₙ₊₁, which are temporally consecutive to each other in the third investigation time period (n is a natural number). The candidate-time detection unit 354 detects a time at which a signal value at a time t reaches minimum as a candidate time. The candidate-time detection unit 354 outputs the detected candidate time (also referred to as a third candidate time) to the output unit 337.

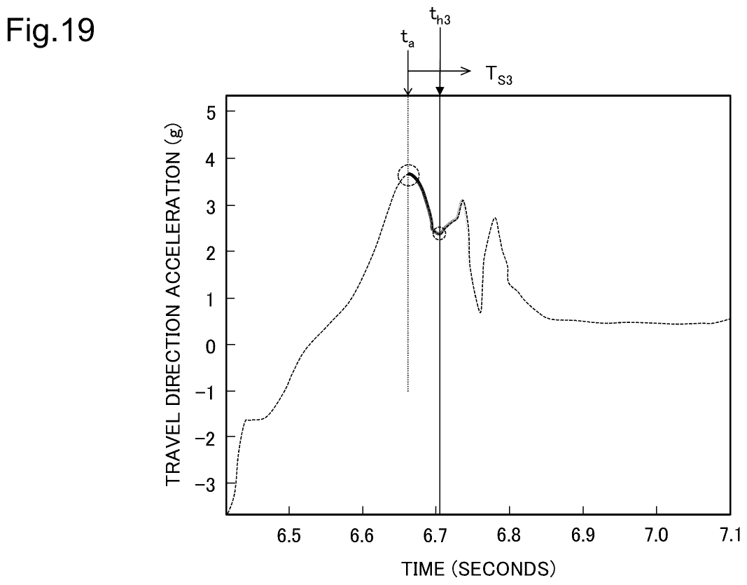

FIG. 19 is a graph for describing an example of detecting a third candidate time tₕ₃ from a waveform of time-series data of travel direction acceleration. The candidate-time detection unit 354 detects, as a third candidate time tₕ₃, a time at which the travel direction acceleration first takes a minimum value in a third investigation time period Tₛ₃ after the acceleration peak time tₐ.

The output unit 337 acquires the third candidate time from the candidate-time detection unit 354. The output unit 337 outputs the acquired third candidate time as a heel strike time. For example, the output unit 337 outputs a heel strike time to a non-illustrated system or device. For example, the output unit 337 outputs a heel strike time to another piece of software installed inside a terminal device on which the detection device 33 has been mounted. For example, the output unit 337 outputs a heel strike time from a terminal device on which the detection device 33 has been mounted to a non-illustrated system or device executed in a cloud or a server. There is no limitation in destination of outputting a heel strike time.

### (Operation)

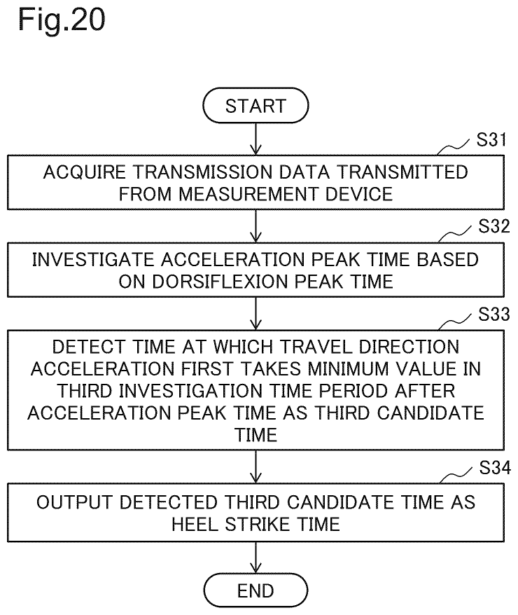

Next, operation of the detection device 33 will now be described herein with reference to the accompanying drawings. FIG. 20 is a flowchart for describing an example of operation of the detection device 33. In the description with reference to the flowchart illustrated in FIG. 20, the detection device 33 will be described as a main operational body.

In FIG. 20, the detection device 33 first acquires transmission data transmitted from the measurement device (not illustrated) (step S31). The transmission data includes travel direction acceleration and a dorsiflexion peak time.

Next, the detection device 33 detects an acceleration peak time based on the dorsiflexion peak time in a waveform of time-series data of the travel direction acceleration (step S32). For example, the detection device 33 detects an acceleration peak time within a time range of approximately 10% of a gait cycle based on a dorsiflexion peak time. For example, the detection device 33 detects an acceleration peak time within a data range corresponding to several samples based on a dorsiflexion peak time.

Next, the detection device 33 detects, as a third candidate time, a time at which the travel direction acceleration first takes a minimum value in the third investigation time period after a maximum acceleration peak time (step S33).

Next, the detection device 33 outputs the detected third candidate time as a heel strike time (step S34). The heel strike time outputted from the detection device 33 is used for detecting a gait event and estimating a physical condition of the user, for example.

As described above, the detection device according to the present example embodiment includes the data acquisition unit, the candidate detection unit, and the output unit. The data acquisition unit acquires data including a dorsiflexion peak time, a plantarflexion peak time, and travel direction acceleration, which are acquired from sensor data regarding a movement of the foot. The candidate detection unit sets a time period starting from the acceleration peak time as a third investigation terminal time period. The candidate detection unit detects, as a candidate time, a time at which the travel direction acceleration first takes an extreme value in the third investigation terminal time period. The output unit outputs the detected candidate time as a heel strike time.

In the present example embodiment, a third investigation time period starting from an acceleration peak time acquired from sensor data measured by the sensor installed on a foot portion of the user is set. In the present example embodiment, a time at which the travel direction acceleration first takes an extreme value in the third investigation terminal time period is detected as a candidate time. According to the present example embodiment, it is possible to uniquely detect heel strike in a gait of the user using data measured by the sensor installed on the foot portion of the user.

### Fourth Example Embodiment

Next, a detection device according to a fourth example embodiment will now be described herein in detail with reference to the accompanying drawings. The detection device according to the present example embodiment is different from that according to the first example embodiment in that a plurality of candidates for a timing of heel strike are detected by combining the methods according to the first to third example embodiments, and a heel strike time is determined based on a result of the detection. The detection device according to the present example embodiment acquires transmission data from the measurement device according to the first example embodiment.

The detection device according to the present example embodiment includes the candidate detection units according to the first to third example embodiments each one in number. The number of the candidate detection units included in the detection device according to the present example embodiment is not limited to three. For example, the detection device according to the present example embodiment may include four or more candidate detection units. For example, the detection device according to the present example embodiment may have a configuration in which two of the candidate detection units included in the first to third example embodiments are combined.

### (Configuration)

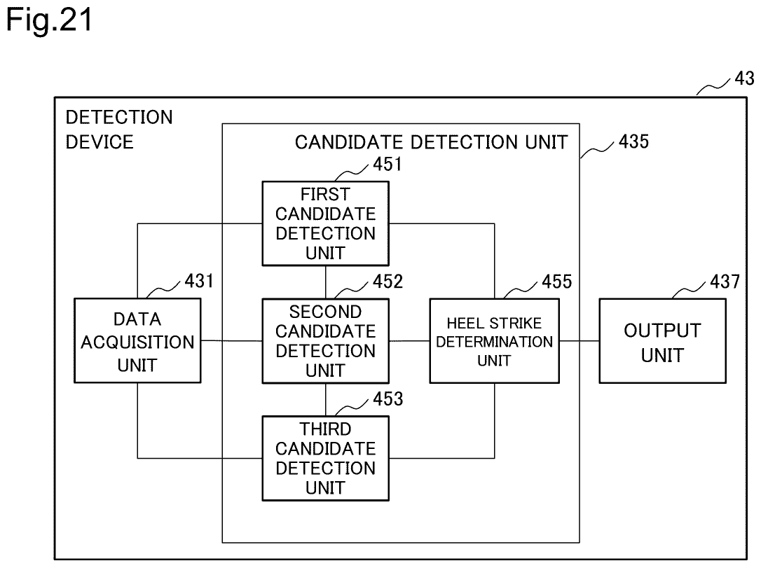

FIG. 21 is a block diagram illustrating an example of a configuration of a detection device 43 according to the present example embodiment. The detection device 43 includes a data acquisition unit 431, a candidate detection unit 435, and an output unit 437. The candidate detection unit 435 includes a first candidate detection unit 451, a second candidate detection unit 452, a third candidate detection unit 453, and a heel strike determination unit 455.

The data acquisition unit 431 has a similar or identical configuration to that of the data acquisition unit 131 according to the first example embodiment. The data acquisition unit 431 acquires transmission data from the measurement device (not illustrated). The data acquisition unit 431 outputs travel direction acceleration, a dorsiflexion peak time, and a plantarflexion peak time included in the acquired transmission data to the first candidate detection unit 451, the second candidate detection unit 452, and the third candidate detection unit 453. The travel direction acceleration includes time-series data of values at signal points at measurement timings (times) for the sensor data. Data to be outputted to each of the first candidate detection unit 451, the second candidate detection unit 452, and the third candidate detection unit 453 will be described later.

The first candidate detection unit 451 has a similar or identical configuration to that of the candidate detection unit 135 according to the first example embodiment. The first candidate detection unit 451 acquires the travel direction acceleration, the dorsiflexion peak time, and the plantarflexion peak time from the data acquisition unit 431. The first candidate detection unit 451 detects an acceleration peak time at which the travel direction acceleration reaches maximum based on the dorsiflexion peak time. The first candidate detection unit 451 detects, as a first investigation terminal time, a time in a mid-stance period at a midpoint between the dorsiflexion peak time and the plantarflexion peak time. The first candidate detection unit 451 detects a first candidate (a first candidate time) for a heel strike time in a first investigation time period between the acceleration peak time and the first investigation terminal time. The first candidate detection unit 451 outputs the detected first candidate time to the heel strike determination unit 455.

The second candidate detection unit 452 has a similar or identical configuration to that of the candidate detection unit 235 according to the second example embodiment. The second candidate detection unit 452 acquires the travel direction acceleration, the dorsiflexion peak time, and the plantarflexion peak time from the data acquisition unit 431. The second candidate detection unit 452 detects an acceleration peak time at which the travel direction acceleration reaches maximum based on the dorsiflexion peak time. The second candidate detection unit 452 detects, as an investigation terminal time, a time in a mid-stance period at a midpoint between the dorsiflexion peak time and the plantarflexion peak time. The second candidate detection unit 452 calculates, as a gait cycle, a period of time between the times in the consecutive mid-stance periods. The second candidate detection unit 452 calculates, as a second investigation terminal time, a time after a predetermined ratio of the gait cycle from the acceleration peak time. The second candidate detection unit 452 detects a second candidate (a second candidate time) for a heel strike time in a second investigation time period between the acceleration peak time and the second investigation terminal time. The second candidate detection unit 452 outputs the detected second candidate time to the heel strike determination unit 455.

The third candidate detection unit 453 has a similar or identical configuration to that of the candidate detection unit 335 according to the third example embodiment. The third candidate detection unit 453 acquires the travel direction acceleration and the dorsiflexion peak time from the data acquisition unit 431. The third candidate detection unit 453 detects an acceleration peak time at which the travel direction acceleration reaches maximum based on the dorsiflexion peak time. The third candidate detection unit 453 detects, as a third candidate time, a time at which the travel direction acceleration first takes a minimum value in a third investigation time period after the acceleration peak time. The third candidate detection unit 453 outputs the detected third candidate time to the heel strike determination unit 455.

The heel strike determination unit 455 acquires the first candidate time from the first candidate detection unit 451. The heel strike determination unit 455 acquires the second candidate time from the second candidate detection unit 452. The heel strike determination unit 455 acquires the third candidate time from the third candidate detection unit 453. The heel strike determination unit 455 determines a heel strike time using the first candidate time, the second candidate time, and the third candidate time.

For example, the heel strike determination unit 455 calculates a weighted average value of the first candidate time, the second candidate time, and the third candidate time as a heel strike time. For example, the heel strike determination unit 455 calculates a weighted average value (a heel strike time tₕ) of a first candidate time tₕ₁, a second candidate time tₕ₂, and a third candidate time tₕ₃ using Formula 1 described below.

$$\begin{matrix}[ Mathematical Formula 1 ] & \\ {t}_{h} = \frac{{a}_{1} \times {t}_{h 1} + {a}_{2} \times {t}_{h 2} + {a}_{3} \times {t}_{h 3}}{{a}_{1} + {a}_{2} + {a}_{3}} & ( 1 )\end{matrix}$$

In Formula 1 described above, a weighting factor set in advance for the first candidate time tₕ₁ is represented by a₁. A weighting factor set in advance for the second candidate time tₕ₂ is represented by a₂. A weighting factor set in advance for the third candidate time tₕ₃ is represented by a₃.

For example, a weighting factor set for each of a first candidate time, a second candidate time, and a third candidate time is set based on an accurate heel strike time measured using motion capture. For example, a weighting factor for a candidate time calculated with each of the detection methods is set in accordance with a result of evaluation on accuracy of a candidate time detected with each of the detection methods for the first candidate detection unit 451, the second candidate detection unit 452, and the third candidate detection unit 453. The smaller the difference from an accurate heel strike time, the higher the accuracy of a candidate time. A weighting factor is set to a larger value as the accuracy of a candidate time calculated with each of the detection methods is higher.

The heel strike determination unit 455 may calculate a statistical value other than a weighted average value as a heel strike time. For example, the heel strike determination unit 455 may calculate an addition average value or a median value of a first candidate time, a second candidate time, and a third candidate time as a heel strike time. For example, the heel strike determination unit 455 may calculate a heel strike time using machine learning based on those including a linear regression model, a support vector machine, and a neural network.

The output unit 437 outputs the heel strike time determined by the heel strike determination unit 455. For example, the output unit 437 outputs the determined heel strike time to a non-illustrated system or device. For example, the output unit 437 outputs a heel strike time to another piece of software installed inside a terminal device on which the detection device 43 has been mounted. For example, the output unit 437 outputs a heel strike time from a terminal device on which the detection device 43 has been mounted to a non-illustrated system or device executed in a cloud or a server.

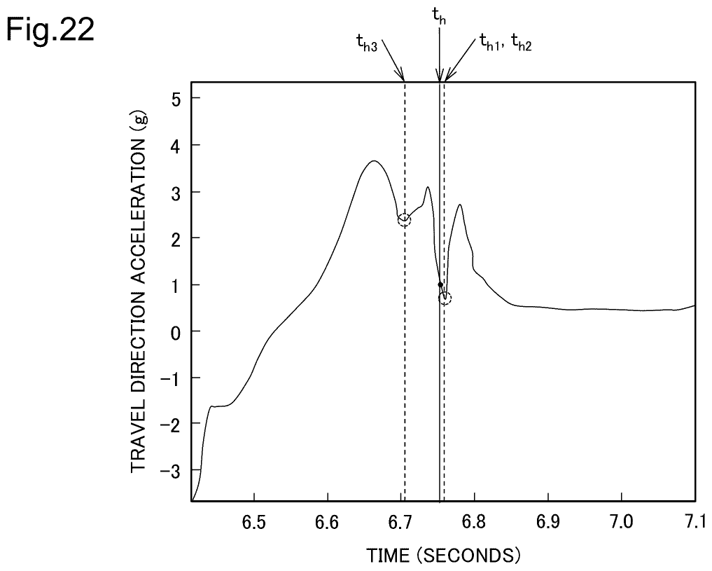

FIG. 22 is a graph for describing an example of determining a heel strike time by the heel strike determination unit 455. The heel strike determination unit 455 calculates a weighted average value acquired by multiplying each of the first candidate time tₕ₁, the second candidate time tₕ₂, and the third candidate time tₕ₃ by a predetermined weight as a heel strike time tₕ. For example, the heel strike determination unit 455 calculates a heel strike time tₕ using Formula 2 described below.

$$\begin{matrix}[ Mathematical Formula 2 ] & \\ {t}_{h} = 0.5 \times {t}_{h 1} + 0.3 \times {t}_{h 2} + 0.2 \times {t}_{h 3} & ( 2 )\end{matrix}$$

In Formula 2 described above, a weight for the first candidate time tₕ₁ is 0.5, a weight for the second candidate time tₕ₂ is 0.3, and a weight for the third candidate time tₕ₃ is 0.2.

### (Operation)

Next, operation of the detection device 43 will now be described herein with reference to the accompanying drawings. FIG. 23 is a flowchart for describing an example of operation of the detection device 43. In the description with reference to the flowchart illustrated in FIG. 23, the detection device 43 will be described as a main operational body.

In FIG. 23, the detection device 43 first acquires transmission data transmitted from the measurement device (not illustrated) (step S41). The transmission data includes travel direction acceleration, a dorsiflexion peak time, and a plantarflexion peak time.

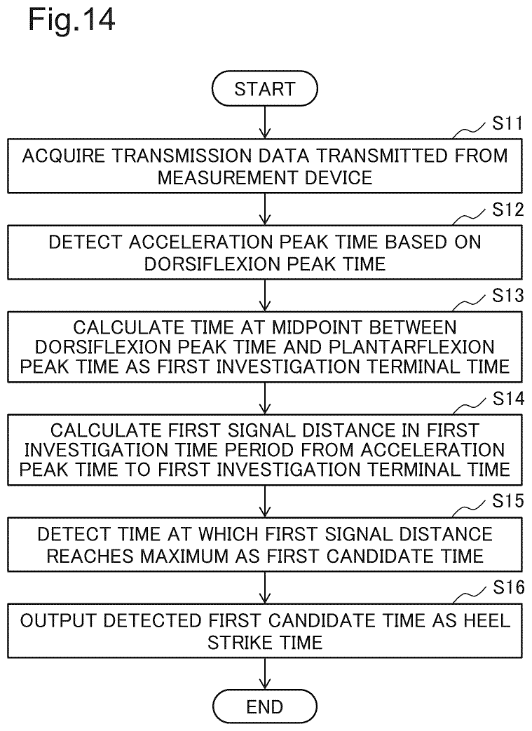

Next, the detection device 43 executes first candidate-detection processing and detects a first candidate time (step S42). The first candidate-detection processing (step S42) is processing (steps S12 to S15 illustrated in FIG. 14) in the detection device 13 according to the first example embodiment.

Next, the detection device 43 executes second candidate-detection processing and detects a second candidate time (step S43). The second candidate-detection processing (step S43) is processing (steps S22 to S27 illustrated in FIG. 17) in the detection device 23 according to the second example embodiment.

Next, the detection device 43 executes third candidate-detection processing and detects a third candidate time (step S44). The third candidate-detection processing (step S44) is processing (steps S32 to S33 illustrated in FIG. 20) in the detection device 33 according to the third example embodiment.

Next, the detection device 43 determines a heel strike time using the detected first candidate time, second candidate time, and third candidate time (step S45). For example, the detection device 43 determines a weighted average value of the first candidate time, the second candidate time, and the third candidate time as a heel strike time.

Next, the detection device 43 outputs the determined heel strike time (step S46). The heel strike time outputted from the detection device 43 is used for detecting a gait event and estimating a physical condition of the user, for example.

As described above, the detection device according to the present example embodiment includes the data acquisition unit, the candidate detection unit, and the output unit. The data acquisition unit acquires data including a dorsiflexion peak time, a plantarflexion peak time, and travel direction acceleration, which are acquired from sensor data regarding a movement of the foot. The candidate detection unit determines a heel strike time in accordance with a condition that has been set in advance from among a plurality of candidate times detected in an investigation time period that has been set for the travel direction acceleration. For example, the candidate detection unit calculates a weighted average value acquired by multiplying each of the plurality of candidate times by a weight that has been set for each of candidate times as a heel strike time. The output unit outputs the determined heel strike time.

In the present example embodiment, a third investigation time period starting from an acceleration peak time acquired from sensor data measured by the sensor installed on a foot portion of the user is set. In the present example embodiment, a time at which the travel direction acceleration first takes an extreme value in the third investigation terminal time period is detected as a candidate time.

In the present example embodiment, a heel strike time is determined using a candidate time detected with a plurality of methods. According to the present example embodiment, it is possible to detect, even when it has been failed to detect heel strike with one of the method, heel strike as long as heel strike is detected with another one of the methods. That is, according to the present example embodiment, it is possible to stably detect heel strike in a gait of the user using candidate times for heel strike, which are detected with a plurality of methods.

### Fifth Example Embodiment

Next, a gait measurement system according to a fifth example embodiment will now be described herein with reference to the accompanying drawings. The gait measurement system according to the present example embodiment includes the configuration of the measurement device according to the first example embodiment. The gait measurement system according to the present example embodiment further includes any one of the configurations of the detection devices according to the first to fourth example embodiments.

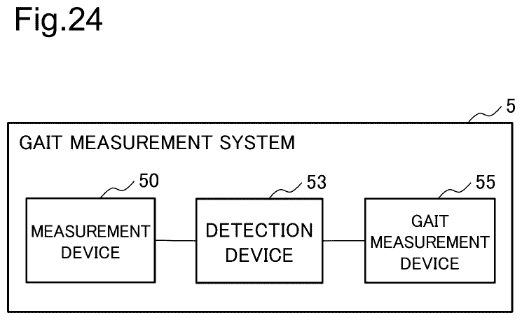

FIG. 24 is a block diagram illustrating an example of a configuration of a gait measurement system 5 according to the present example embodiment. The gait measurement system 5 includes a measurement device 50, a detection device 53, and a gait measurement device 55. The measurement device 50 and the detection device 53 form a detection system.

The measurement device 50 has a similar or identical configuration to that of the measurement device 10 according to the first example embodiment. The measurement device 50 is installed on a foot portion of the user. The measurement device 50 measures sensor data regarding a movement of the foot. The measurement device 50 includes a sensor including an acceleration sensor and an angular velocity sensor, for example. The measurement device 50 generates sensor data using a measurement value measured by the sensor in accordance with a movement of the foot. The measurement device 50 smooths travel direction acceleration. The measurement device 50 detects a dorsiflexion peak time and a plantarflexion peak time from the measured sensor data. The measurement device 50 outputs, to the detection device 53, transmission data including the smoothed travel direction acceleration (the travel direction acceleration), the dorsiflexion peak time, and the plantarflexion peak time.

The detection device 53 has a configuration similar or identical to any one of those of the detection devices according to the first to fourth example embodiments. The detection device 53 acquires the transmission data from the measurement device 50. The detection device 53 detects a candidate time for a heel strike time using the travel direction acceleration, the dorsiflexion peak time, and the plantarflexion peak time included in the acquired transmission data. The detection device 53 outputs a heel strike time corresponding to the detected candidate time to the gait measurement device 55. The detection device 53 may output times such as a dorsiflexion peak time, a plantarflexion peak time, a time in a mid-stance period, and an acceleration peak time. For example, it is possible to use a time in a mid-stance period as a reference for extracting time-series data of sensor data corresponding to one gait cycle.

The gait measurement device 55 acquires the heel strike time from the detection device 53. The gait measurement device 55 performs other tasks including detection of a gait event and calculation of gait parameters, for example, using the acquired heel strike time. For example, the gait measurement device 55 estimates a physical condition of the user using calculated gait parameters. The gait measurement device 55 outputs information about a timing of the detected gait event, the calculated gait parameters, and the estimated physical information, for example. Detailed description of information to be outputted from the gait measurement device 55 is omitted.

For example, the gait measurement device 55 detects, based on a heel strike time, gait events such as separation of toe of opposite foot from ground, heel rising, grounding of heel of opposite foot, separation of toe from ground, crossing feet, and vertical tibia. For example, the gait measurement device 55 detects a gait event from time-series data (also referred to as a gait waveform) of sensor data corresponding to one gait cycle starting from a time in a mid-stance period. For example, the gait measurement device 55 detects a gait event in accordance with features appearing in a gait waveform, such as travel direction acceleration, vertical direction acceleration, a roll angular velocity, and a roll angle.

For example, the gait measurement device 55 may specify a characteristic section (also referred to as a gait period) included in an evaluation-target section based on a gait event detected from a gait waveform. For example, the gait measurement device 55 specifies, as a load response period, a section between heel strike and separation of toe of opposite foot from ground. For example, the gait measurement device 55 specifies, as a mid-stance period, a section between separation of toe of opposite foot from ground and heel rising. For example, the gait measurement device 55 specifies, as a terminal stance period, a section between heel rising and grounding of heel of opposite foot. For example, the gait measurement device 55 specifies, as a pre-swing period, a section between grounding of heel of opposite foot and separation of toe from ground. For example, the gait measurement device 55 specifies, as an initial swing period, a section between separation of toe from ground and crossing feet. For example, the gait measurement device 55 specifies, as a mid-swing period, a section between crossing feet and vertical tibia. For example, the gait measurement device 55 specifies, as a terminal swing period, a section between vertical tibia and heel strike.

For example, the gait measurement device 55 calculates gait parameters such as a gait speed, a stride, a grounding angle, a ground-separation angle, a foot-raising height, a circumduction gait, and a foot angle, in accordance with a time of a gait event and a time of a gait period. For example, the gait measurement device 55 calculates a gait speed by dividing a movement distance between detection times acquired by performing a second-order integration on travel direction acceleration for identical gait events continuously detected by a time interval of the detection times. For example, the gait measurement device 55 calculates, as a stride, an absolute value of a difference between a space position at a time of crossing feet and a space position at a time of separation of toe from ground for a gait waveform of a travel direction trajectory. For example, the gait measurement device 55 calculates, as a grounding angle, an attitude angle at a heel strike time. For example, the gait measurement device 55 calculates, as a ground-separation angle, an attitude angle at a time of separation of toe from ground. For example, the gait measurement device 55 calculates a maximum foot-raising height based on a trajectory in the sagittal plane, which is acquired by performing a second-order integration on a vertical direction acceleration. For example, the gait measurement device 55 calculates a circumduction gait based on a trajectory in the horizontal plane, which is acquired by performing a second-order integration on left-and-right direction acceleration. For example, the gait measurement device 55 calculates, as a foot angle, an angle formed by a velocity vector and a center line of a foot, using the velocity vector and an attitude angle of the foot portion.

For example, the gait measurement device 55 estimates a physical condition such as gait symmetry, a progress status of hallux valgus, and degrees of pronation and supination of a foot, based on a gait event and gait parameters. For example, the gait measurement device 55 compares extreme values immediately before a heel strike time in time-series data of an attitude angle measured by the measurement devices 50 installed on the left and right feet, and estimates the gait symmetry. For example, the gait measurement device 55 estimates a progress status of hallux valgus using a model having undergone machine learning on feature amounts about hallux valgus, which are extracted from sensor data regarding a movement of the foot. For example, the gait measurement device 55 estimates degrees of pronation and supination of a foot using feature amounts extracted from an angular waveform in the coronal plane during a terminal stance period.

### Application Example

Next, an application example of the gait measurement device 55 according to the present example embodiment will now be described herein with reference to the accompanying drawings. In an example described in here, the function of the gait measurement device 55 installed in a mobile terminal carried by the user estimates a physical condition of the user using feature amount data measured by the measurement device 50 disposed on a shoe.

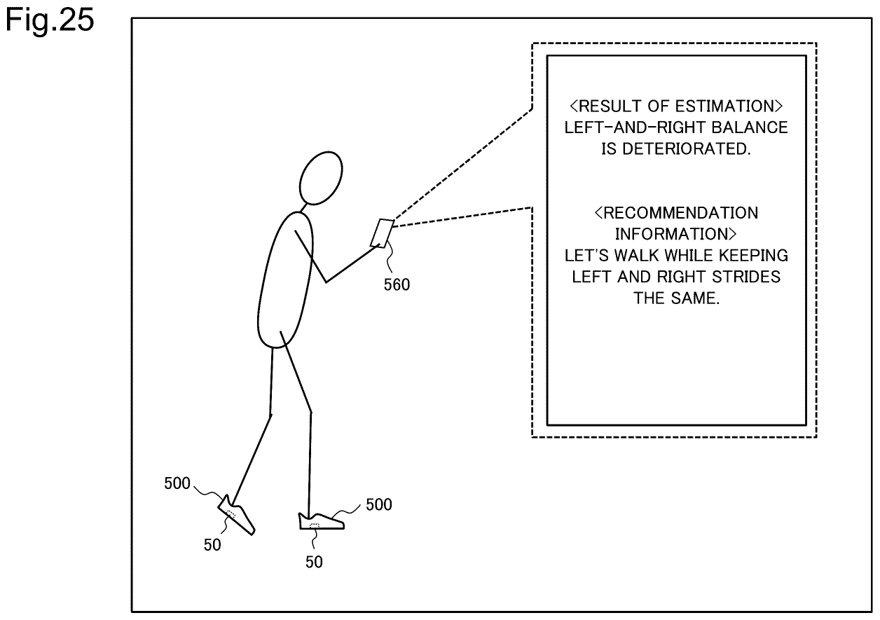

FIG. 25 is a conceptual diagram illustrating an example in which information in accordance with a result of measurement by the gait measurement device 55 is displayed on a screen of a mobile terminal 560 carried by the user walking while wearing shoes 500 on which the measurement devices 50 are disposed, respectively. FIG. 25 illustrates an example in which a result of estimation and recommendation information in accordance with sensor data measured while the user is walking are displayed on the screen of the mobile terminal 560.

For example, the gait measurement device 55 calculates a score quantified based on a criterion that has been set in advance as a result of estimation about a physical condition. The gait measurement device 55 causes the screen of the mobile terminal 560 to display information about a result of estimation on a physical condition “Left-and-right balance is deteriorated.”, in accordance with a score about a physical condition. The gait measurement device 55 causes the screen of the mobile terminal 560 to display recommendation information “Let's walk while keeping left and right strides the same.”, in accordance with the score of the physical condition. As the user confirms the information displayed on the screen of the mobile terminal 560, it is possible to urge the user to walk while being conscious of the left and right strides in accordance with the displayed recommendation information, making it possible to urge the user to practice an exercise for improving the left and right balance.

As described above, the gait measurement system according to the present example embodiment includes the measurement device, the detection device, and the gait measurement device. The measurement device includes the sensor and the peak detection unit. The sensor is installed on footwear of the user. The sensor measures spatial acceleration and a spatial angular velocity. The sensor generates sensor data regarding a movement of the foot using the measured spatial acceleration and spatial angular velocity. The sensor outputs the generated sensor data, and the peak detection unit acquires time-series data of the sensor data. The peak detection unit smooths time-series data of travel direction acceleration, which is included in the sensor data. The peak detection unit detects a dorsiflexion peak time and a plantarflexion peak time from time-series data of a roll angle, which is included in the sensor data. The peak detection unit outputs data including the smoothed travel direction acceleration, the dorsiflexion peak time, and the plantarflexion peak time to the detection device.

The detection device includes the data acquisition unit, the candidate detection unit, and the output unit. The data acquisition unit acquires data including a dorsiflexion peak time, a plantarflexion peak time, and travel direction acceleration, which are acquired from sensor data regarding a movement of the foot. The candidate detection unit detects a time of a feature signal point extracted from time-series data of travel direction acceleration as a candidate time for heel strike in an investigation time period starting from an acceleration peak time detected from the travel direction acceleration based on the dorsiflexion peak time. The output unit outputs the detected candidate time as a heel strike time.

The gait measurement device detects a gait event from the sensor data based on the heel strike time detected by the detection device. The gait measurement device calculates gait parameters in accordance with the detected gait event. The gait measurement device measures a gait of the user using the calculated gait parameters.

With the present example embodiment, it is possible to measure a gait of the user using gait parameters calculated based on a heel strike time detected by the detection device.

### Sixth Example Embodiment

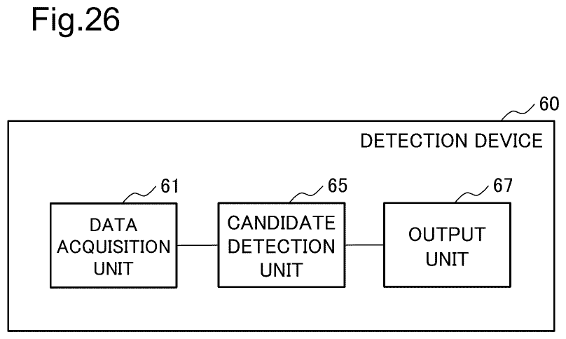

Next, a detection device according to a sixth example embodiment will now be described herein with reference to the accompanying drawings. The detection device according to the present example embodiment has a configuration in which the detection devices according to the first to fifth example embodiments are simplified. FIG. 26 is a block diagram illustrating an example of a configuration of a detection device 60 according to the present example embodiment. The detection device 60 includes a data acquisition unit 61, a candidate detection unit 65, and an output unit 67.

The data acquisition unit 61 acquires data including a dorsiflexion peak time, a plantarflexion peak time, and travel direction acceleration, which are acquired from sensor data regarding a movement of the foot. The candidate detection unit 65 detects a time of a feature signal point extracted from time-series data of travel direction acceleration as a candidate time for heel strike in an investigation time period starting from an acceleration peak time detected from the travel direction acceleration based on the dorsiflexion peak time. The output unit 67 outputs the detected candidate time as a heel strike time.

In the present example embodiment, an investigation time period for heel strike, which starts from an acceleration peak time acquired from sensor data measured by the sensor installed on a foot portion of the user, is set. In the present example embodiment, a candidate time detected in the set investigation time period is detected as a heel strike time. According to the present example embodiment, it is possible to detect heel strike in a gait of the user using data measured by the sensor installed on the foot portion of the user.

### (Hardware Configuration)

A hardware configuration for executing processing according to each of the example embodiments of the present disclosure will now be described herein using an information processing device 90 illustrated in FIG. 30 as an example. The information processing device 90 illustrated in FIG. 30 is a mere configuration example for executing the processing according to each of the example embodiments, and there is no intention of limiting the scope of the present disclosure.

As illustrated in FIG. 30, the information processing device 90 includes a processor 91, a main storage device 92, an auxiliary storage device 93, an input-and-output interface 95, and a communication interface 96. In FIG. 30, I/F means an abbreviation of interface. The processor 91, the main storage device 92, the auxiliary storage device 93, the input-and-output interface 95, and the communication interface 96 are coupled to each other via a bus 98 to achieve data communication. The processor 91, the main storage device 92, the auxiliary storage device 93, and the input-and-output interface 95 are coupled to a network such as the Internet or an intranet via the communication interface 96.

The processor 91 deploys, in the main storage device 92, a program stored in the auxiliary storage device 93 or another storage. The processor 91 executes the program deployed in the main storage device 92. In the present example embodiment, a software program installed in the information processing device 90 may be used. The processor 91 executes processing according to each of the example embodiments.

The main storage device 92 has a region in which a program is to be deployed. A program stored in the auxiliary storage device 93 or another storage is deployed in the main storage device 92 by the processor 91. The main storage device 92 is achieved by, for example, a volatile memory such as a dynamic random access memory (DRAM). A non-volatile memory such as a magnetoresistive random access memory (MRAM) may be formed or added as the main storage device 92.

The auxiliary storage device 93 stores various types of data including programs, for example. The auxiliary storage device 93 is achieved by a local disk such as a hard disk or a flash memory. Various types of data may be stored in the main storage device 92, and the auxiliary storage device 93 may be omitted.

The input-and-output interface 95 serves as an interface for coupling the information processing device 90 and a peripheral device based on a standard or a specification. The communication interface 96 serves as an interface for coupling to an external system or device through a network such as the Internet or an intranet based on a standard or a specification. The input-and-output interface 95 and the communication interface 96 may be formed as a common interface coupled to an external device.

Input devices such as a keyboard, a mouse, and a touch panel, for example, may be coupled to the information processing device 90 as necessary. These input devices are used to input information and settings. When a touch panel is used as an input device, a display screen of a display device may also serve as an interface for the input device. Data communication between the processor 91 and the input devices may be mediated by the input-and-output interface 95.

The information processing device 90 may be provided with a display device for displaying information. When a display device is provided, it is preferable that the information processing device 90 be provided with a display control device (not illustrated) for controlling display of the display device. The display device may be coupled to the information processing device 90 via the input-and-output interface 95.

The information processing device 90 may be provided with a drive device. The drive device mediates, between the processor 91 and a recording medium (a program recording medium), reading of data and a program from the recording medium and writing of a result of processing in the information processing device 90 to the recording medium, for example. The drive device may be coupled to the information processing device 90 via the input-and-output interface 95.

The example of the hardware configuration for enabling processing according to each of the example embodiments of the present invention has been described above. The hardware configuration illustrated in FIG. 30 is a mere example of a hardware configuration for executing processing according to each of the example embodiments, and there is no intention of limiting the scope of the present invention. A program for causing a computer to execute processing according to each of the example embodiments is also included in the scope of the present invention. A program recording medium in which a program according to each of the example embodiments is recorded is also included in the scope of the present invention. It is possible to achieve the recording medium by, for example, an optical recording medium such as a compact disc (CD) or a digital versatile disc (DVD). The recording medium may be achieved by a semiconductor recording medium such as a universal serial bus (USB) memory or a secure digital (SD) card. The recording medium may be achieved by a magnetic recording medium such as a flexible disk or another recording medium. When a program to be executed by the processor has been recorded in a recording medium, the recording medium corresponds to a program recording medium.

The components of each of the example embodiments may be combined in a desired manner. The components of each of the example embodiments may be achieved by software or may be achieved by a circuit.

While the present invention has been particularly shown and described with reference to the example embodiments thereof, the invention is not limited to these example embodiments. It will be understood by those of ordinary skill in the art that various changes in form and details may be made therein without departing from the spirit and scope of the present invention as defined by the claims.

### REFERENCE SIGNS LIST

1 detection system
5 gait measurement system
10, 50 measurement device
11 sensor
12 peak detection unit
13 detection device
111 acceleration sensor
112 angular velocity sensor
121 coordinate conversion unit
122 low-pass filter
123 roll-angle calculation unit
124 plantar-angle-peak detection unit
125 dorsiflexion-peak detection unit
126 plantarflexion-peak detection unit
127 data transmission unit
131, 231, 331 data acquisition unit
135, 235, 335 candidate detection unit
137, 237, 337 output unit
150, 250 investigation-time-period setting unit
151, 251, 351 acceleration-peak-time detection unit
152, 252 investigation-terminal-time calculation unit
153, 253 signal-distance calculation unit
154, 254, 354 candidate-time detection unit

What is claimed is:

1. A detection device comprising:
a memory storing instructions; and
a processor connected to the memory and configured to execute the instructions to:
acquire data including a dorsiflexion peak time, a plantarflexion peak time, and travel direction acceleration acquired from sensor data regarding a movement of a foot;
detect, as a candidate time for heel strike, a time of a feature signal point extracted from time-series data of the travel direction acceleration in an investigation time period starting from an acceleration peak time detected from the travel direction acceleration based on the dorsiflexion peak time; and
output the detected candidate time as a heel strike time.

2. The detection device according to claim 1, wherein
the processor is configured to execute the instructions to
calculate, as a first investigation terminal time, a time in a mid-stance period, the time in the mid-stance period corresponding to a time at a midpoint between the dorsiflexion peak time and the plantarflexion peak time,
set a time period from the acceleration peak time to the first investigation terminal time as a first investigation time period,
calculate, for a signal point of the travel direction acceleration included in the first investigation time period, a first signal distance corresponding to a Euclidean distance of a signal point of the travel direction acceleration with respect to a first reference straight line passing through a signal point of the travel direction acceleration at the acceleration peak time and a signal point of the travel direction acceleration at the first investigation terminal time, and
detect a time of the feature signal point at which the calculated first signal distance takes a maximum value as the candidate time.

3. The detection device according to claim 1, wherein
the processor is configured to execute the instructions to
calculate a time in a mid-stance period, the time in the mid-stance period corresponding to a time at a midpoint between the dorsiflexion peak time and the plantarflexion peak time,
calculate, as one gait cycle, a time period between the times in the consecutive mid-stance periods,
set a time after a predetermined ratio of the one gait cycle from the acceleration peak time as a second investigation terminal time,
set a time period from the acceleration peak time to the second investigation terminal time as a second investigation time period,
calculate, for a signal point of the travel direction acceleration included in the second investigation time period, a second signal distance corresponding to a Euclidean distance of a signal point of the travel direction acceleration with respect to a second reference straight line passing through a signal point of the travel direction acceleration at the acceleration peak time and a signal point of the travel direction acceleration at the second investigation terminal time, and
detect a time of the feature signal point at which the calculated second signal distance takes a maximum value as the candidate time.

4. The detection device according to claim 1, wherein
the processor is configured to execute the instructions to
set a time period starting from the acceleration peak time as a third investigation terminal time period, and
detect a time at which the travel direction acceleration first takes an extreme value in the third investigation terminal time period as the candidate time.

5. The detection device according to claim 1, wherein
the processor is configured to execute the instructions to
determine the heel strike time in accordance with a condition that has been set in advance from among a plurality of the candidate times detected in the investigation time period that has been set for the travel direction acceleration.

6. The detection device according to claim 5, wherein
the processor is configured to execute the instructions to
calculate a weighted average value acquired by multiplying each of the plurality of candidate times by a weight that has been set for each of the candidate times as the heel strike time.

7. A detection system comprising:
the detection device according to claim 1; and
a measurement device including
a sensor that is installed on footwear of a user, measures spatial acceleration and a spatial angular velocity, generates sensor data regarding a movement of a foot using the measured spatial acceleration and the measured spatial angular velocity, and outputs the generated sensor data,
a memory storing instructions; and
a processor connected to the memory and configured to execute the instructions to
acquire time-series data of the sensor data,
smooth time-series data of travel direction acceleration, the time-series data of the travel direction acceleration being included in the sensor data,
detect a dorsiflexion peak time and a plantarflexion peak time from time-series data of a roll angle, the time-series data of the roll angle being included in the sensor data, and
output data including the smoothed travel direction acceleration, the dorsiflexion peak time, and the plantarflexion peak time to the detection device.

8. A gait measurement system comprising:
the detection device according to claim 1;
a measurement device including
a sensor that is installed on footwear of a user, measures spatial acceleration and a spatial angular velocity, generates sensor data regarding a movement of a foot using the measured spatial acceleration and the measured spatial angular velocity, and outputs the generated sensor data, and
a memory storing instructions; and
a processor connected to the memory and configured to execute the instructions to
acquire time-series data of the sensor data,
smooth time-series data of travel direction acceleration, the time-series data of the travel direction acceleration being included in the sensor data,
detect a dorsiflexion peak time and a plantarflexion peak time from time-series data of a roll angle, the time-series data of the roll angle being included in the sensor data, and
output data including the smoothed travel direction acceleration, the dorsiflexion peak time, and the plantarflexion peak time to the detection device; and
a gait measurement device including
a memory storing instructions; and
a processor connected to the memory and configured to execute the instructions to
detect a gait event from the sensor data based on a heel strike time detected by the detection device,
calculate a gait parameter in accordance with the detected gait event, and
measure a gait of the user using the calculated gait parameter.

9. A detection method comprising causing a computer to
acquire data including a dorsiflexion peak time, a plantarflexion peak time, and travel direction acceleration acquired from sensor data regarding a movement of a foot,
detect, as a candidate time for heel strike, a time of a feature signal point extracted from time-series data of the travel direction acceleration in an investigation time period starting from an acceleration peak time detected from the travel direction acceleration based on the dorsiflexion peak time, and
output the detected candidate time as a heel strike time.

10. A non-transitory recording medium recording a program causing a computer to execute
processing for acquiring data including a dorsiflexion peak time, a plantarflexion peak time, and travel direction acceleration acquired from sensor data regarding a movement of a foot,
processing for detecting, as a candidate time for heel strike, a time of a feature signal point extracted from time-series data of the travel direction acceleration in an investigation time period starting from an acceleration peak time detected from the travel direction acceleration based on the dorsiflexion peak time, and
processing for outputting the detected candidate time as a heel strike time.

11. The gait measurement system according to claim 1, wherein
the processor included in the gait measurement device is configured to execute the instructions to
calculate the heel strike time using machine learning,
calculate a gait parameter using the heel strike time,
estimate a physical condition of the user using the gait parameter, and
output recommendation information that supports the user for making decision about taking an action.
